SEQUENCE LISTING (54 sequences)

SEQ ID NO: 1 - DNA, 18 bp, Artificial Sequence
  Description of Artificial Sequence Synthetic primer
gggccctgaaggctgtag

SEQ ID NO: 2 - DNA, 18 bp, Artificial Sequence
  Description of Artificial Sequence Synthetic primer
aatggagccactgccatc

SEQ ID NO: 3 - DNA, 18 bp, Artificial Sequence
  Description of Artificial Sequence Synthetic primer
gggccctgaaggctgtag

SEQ ID NO: 4 - DNA, 18 bp, Artificial Sequence
  Description of Artificial Sequence Synthetic primer
aatggagccactgccatc

SEQ ID NO: 5 - DNA, 18 bp, Artificial Sequence
  Description of Artificial Sequence Synthetic primer
aacacggctcgatgctgg

SEQ ID NO: 6 - DNA, 18 bp, Artificial Sequence
  Description of Artificial Sequence Synthetic primer
ccagctgccacttgatgc

SEQ ID NO: 7 - DNA, 19 bp, Artificial Sequence
  Description of Artificial Sequence Synthetic primer
cccagcaaaccttctacca

SEQ ID NO: 8 - DNA, 19 bp, Artificial Sequence
  Description of Artificial Sequence Synthetic primer
aagcagccaggtactcagg

SEQ ID NO: 9 - DNA, 32 bp, Artificial Sequence
  Description of Artificial Sequence Synthetic primer
caccatggattacaaggatgacgacgataagg

SEQ ID NO: 10 - DNA, 36 bp, Artificial Sequence
  Description of Artificial Sequence Synthetic primer
ttacaaggggttgctgagctgttctgatttggttcc

SEQ ID NO: 11 - DNA, 50 bp, Artificial Sequence
  Description of Artificial Sequence Synthetic oligonucleotide
caccgatggattgcacgcaggttctcgaaagaacctgcgtgcaatccatc

SEQ ID NO: 12 - DNA, 50 bp, Artificial Sequence
  Description of Artificial Sequence Synthetic oligonucleotide
aaaagatggattgcacgcaggttctttcgagaacctgcgtgcaatccatc

SEQ ID NO: 13 - DNA, 50 bp, Artificial Sequence
  Description of Artificial Sequence Synthetic oligonucleotide
caccgggattattggagctcctttccgaagaaaggagctccaataatccc

SEQ ID NO: 14 - DNA, 50 bp, Artificial Sequence
  Description of Artificial Sequence Synthetic oligonucleotide
aaaagggattattggagctcctttcttcggaaaggagctccaataatccc

SEQ ID NO: 15 - DNA, 50 bp, Artificial Sequence
  Description of Artificial Sequence Synthetic oligonucleotide
caccggagacaaagctaccacatgtcgaaacatgtggtagctttgtctcc

SEQ ID NO: 16 - DNA, 50 bp, Artificial Sequence
  Description of Artificial Sequence Synthetic oligonucleotide
caccggttctttagctgtcacttagcgaactaagtgacagctaaagaacc

SEQ ID NO: 17 - DNA, 50 bp, Artificial Sequence
  Description of Artificial Sequence Synthetic oligonucleotide
aaaaggttctttagctgtcacttagttcgctaagtgacagctaaagaacc

SEQ ID NO: 18 - DNA, 50 bp, Artificial Sequence
  Description of Artificial Sequence Synthetic oligonucleotide
caccgcattccatcctgaagaaatccgaagatttcttcaggatggaatgc

SEQ ID NO: 19 - DNA, 50 bp, Artificial Sequence
  Description of Artificial Sequence Synthetic oligonucleotide
aaaagcattccatcctgaagaaatcttcggatttcttcaggatggaatgc

SEQ ID NO: 20 - DNA, 50 bp, Artificial Sequence
  Description of Artificial Sequence Synthetic oligonucleotide
caccgcagatgcagagatttaatgtcgaaacattaaatctctgcatctgc

SEQ ID NO: 21 - DNA, 50 bp, Artificial Sequence
  Description of Artificial Sequence Synthetic oligonucleotide
aaaagcagatgcagagatttaatgtttcgacattaaatctctgcatctgc

SEQ ID NO: 22 - DNA, 50 bp, Artificial Sequence
  Description of Artificial Sequence Synthetic oligonucleotide
caccggagaaggaggtcgaagaaatcgaaatttcttcgacctccttctcc

SEQ ID NO: 23 - DNA, 50 bp, Artificial Sequence
  Description of Artificial Sequence Synthetic oligonucleotide
aaaaggagaaggaggtcgaagaaatttcgatttcttcgacctccttctcc

SEQ ID NO: 24 - DNA, 36 bp, Artificial Sequence
  Description of Artificial Sequence Synthetic primer
gggctcgagtgaggtgaaataaatttcaggagttta

SEQ ID NO: 25 - DNA, 33 bp, Artificial Sequence
  Description of Artificial Sequence Synthetic primer
gccaagcttagccgggagtgagcgccaccgccc

SEQ ID NO: 26 - DNA, 33 bp, Artificial Sequence
  Description of Artificial Sequence Synthetic primer
gggctcgagataaccagcgctcccgttattcag

SEQ ID NO: 27 - DNA, 33 bp, Artificial Sequence; identical to SEQ ID NO: 25
  Description of Artificial Sequence Synthetic primer

SEQ ID NO: 28 - DNA, 33 bp, Artificial Sequence
  Description of Artificial Sequence Synthetic primer
gggctcgaggttcaggaacctggcatgggccgg

SEQ ID NO: 29 - DNA, 33 bp, Artificial Sequence; identical to SEQ ID NO: 25
  Description of Artificial Sequence Synthetic primer

SEQ ID NO: 30 - DNA, 36 bp, Artificial Sequence; identical to SEQ ID NO: 24
  Description of Artificial Sequence Synthetic primer

SEQ ID NO: 31 - DNA, 33 bp, Artificial Sequence
  Description of Artificial Sequence Synthetic primer
gccaagcttccggcccatgccaggttcctgaac

SEQ ID NO: 32 - DNA, 1000 bp, Homo sapiens
tgaggtgaaataaatttcaggagtttatgtaaaaaattaatttggtagaaataatgtctagattcttgcatgccatggtgtcttggttaagaacttggaatgtggagtcagctctgccacttacaagctatgtggctctgggaaaattacttatgtctttgatcttttcttcgctttaaaaagtaacctgtggtatagaattggaggggtaaagttcttggaatgctgagattattaaattattattcgttattaaaactctagttacttagcacttacagctctttagcttagaaatagtctaaatacgttgctatatttgcttttttaataaccagcgctcccgttattcaggtaaagaattaaaaacatctaaaaggtaattgctttgtaaatcctaacctctttcattcaaaatacctaaggtagatggctctttataaacgtgcccggctcagagagctctcatctatattatatctccgaatccaaatttcactgatgcccagtctaagagtgtttcctgggctttgagctttgagatagaacgaagcaggtggatcctggctttgactaggcagggccagaccaggcccgacccagccagcccatccgtagagtcagcatcagccgggcgtgaagattggtgtcggctcctgcgttccgccaagttcaggaacctggcatgggccggccgcctcccgcgaagggacacacctgttggtcaccgcgctcttactggccagacccggatattggccttctggcgtcagcggcaggggcatgtccctggccggagagcacaggccagagtggggtgacgtcatcgaaggcacgtcccagccttgcagagcggtgggcggggcctgcgaagaaggtgtgccgggggctggttggaggcgcgggcggcggggccaatgtggcggtgacgtcacggggcgggcggacgctggcgcgggtaggtaagagcagcggcgggcggtggcgctcactcccggct

SEQ ID NO: 33 - DNA, 15 bp, Homo sapiens
gaaataaatttcagg

SEQ ID NO: 34 - DNA, 16 bp, Homo sapiens
tttatgtaaaaaatta

SEQ ID NO: 35 - DNA, 8 bp, Homo sapiens
ttatgtaa

SEQ ID NO: 36 - DNA, 21 bp, Homo sapiens
ttatgtaaaaaattaatttgg

SEQ ID NO: 37 - DNA, 7 bp, Homo sapiens
ctttgta

SEQ ID NO: 38 - DNA, 6 bp, Homo sapiens
tttata

SEQ ID NO: 39 - DNA, 6 bp, Homo sapiens
acgtgc

SEQ ID NO: 40 - DNA, 5 bp, Homo sapiens
attat

SEQ ID NO: 41 - DNA, 20 bp, Homo sapiens
tgactaggcagggccagacc

SEQ ID NO: 42 - DNA, 8 bp, Homo sapiens
cagcggca

SEQ ID NO: 43 - DNA, 7 bp, Homo sapiens
caggggc

SEQ ID NO: 44 - DNA, 15 bp, Homo sapiens
gagtggggtgacgtc

SEQ ID NO: 45 - DNA, 12 bp, Homo sapiens
ggtgacgtcatc

SEQ ID NO: 46 - DNA, 8 bp, Homo sapiens
tgacgtca

SEQ ID NO: 47 - DNA, 10 bp, Homo sapiens
tgggcggggc

SEQ ID NO: 48 - DNA, 10 bp, Homo sapiens
gcgcgggcgg

SEQ ID NO: 49 - DNA, 10 bp, Homo sapiens
ggcggcgggg

SEQ ID NO: 50 - DNA, 7 bp, Homo sapiens
gcggggc

SEQ ID NO: 51 - DNA, 12 bp, Homo sapiens
ggtgacgtcacg

SEQ ID NO: 52 - DNA, 12 bp, Homo sapiens
ggtgacgtcacg

SEQ ID NO: 53 - DNA, 8 bp, Homo sapiens
tgacgtca

SEQ ID NO: 54 - DNA, 50 bp, Artificial Sequence
  Description of Artificial Sequence Synthetic primer
aaaaggagacaaagctaccacatgtttcgacatgtggtagctttgtctcc

(19) United States
(12) Patent Application Publication
(10) Pub. No.: US 2016/0199463 A1
(43) Pub. Date: Jul. 14, 2016

(54) HDAC2 DEFENDS VASCULAR ENDOTHELIUM FROM INJURY

(71) Applicant: The Johns Hopkins University, Baltimore, MD (US)

(72) Inventors: Lewis Romer, Baltimore, MD (US); Deepesh Pandey, Baltimore, MD (US); Dan E. Berkowitz, Baltimore, MD (US)

(21) Appl. No.: 14/969,944

(22) Filed: Dec. 15, 2015

Related U.S. Application Data
(60) Provisional application No. 62/091,938, filed on Dec. 15, 2014.

(51) Int. Cl. A61K 38/50; A61K 31/519; C12N 7/00; C12N 15/113; A61K 31/522; A61K 31/12

(52) U.S. Cl. CPC A61K 38/50; A61K 31/522; A61K 31/12; C12N 7/00; C12N 15/1137; A61K 31/519; A61K 48/00

(57) ABSTRACT

The invention features compositions comprising HDAC2 and methods for protecting the vascular endothelium from injury.

### CROSS-REFERENCE TO RELATED APPLICATIONS

This application claims the benefit of priority under 35 U.S.C. §119(e) to U.S. Provisional Application No. 62/091,938, filed Dec. 15, 2014 which is incorporated herein by reference in its entirety.

### STATEMENT OF RIGHTS TO INVENTIONS MADE UNDER FEDERALLY SPONSORED RESEARCH

This work was supported by the National Heart, Lung, and Blood Institute under grant number HL089668. The Government has certain rights in this invention.

### FIELD OF THE INVENTION

This invention relates generally to the field of therapies for endothelial dysfunction and artherosclerosis.

### BACKGROUND OF THE INVENTION

Atherosclerotic cardiovascular disease is a leading cause of mortality in the Western world. Its pathobiology involves chronic inflammation of the vascular wall, resulting from endothelial dysfunction, adhesion molecule expression, and monocyte infiltration of the intima, ultimately leading to plaque development. As such, prior to the invention described herein, there was a pressing need to develop new strategies for the treatment of atherosclerosis.

### SUMMARY OF THE INVENTION

The invention is based, at least in part, on the surprising discovery that histone deacetylase 2 (HDAC2) tonically limits the availability of the endothelial nitric oxide synthase (eNOS) competitor arginase 2 (Arg2) in vascular endothelium by constraining Arg2 transcription in baseline homeostatic conditions, and that this brake on Arg2 population in human aortic endothelial cells (HAECs) is released by oxidative injury. Described herein are therapeutic interventions that restore and preserve vascular endothelial health against the epidemic onslaught of oxidative injury and atherogenesis.

Described herein are methods of treating or preventing endothelial dysfunction or an endothelial dysfunction-associated condition in a subject. First, a subject having or at risk of developing endothelial dysfunction or an endothelial dysfunction-associated condition is identified. The subject is preferably a mammal in need of such treatment, e.g., a subject that has been diagnosed with endothelial dysfunction or an endothelial dysfunction-associated condition or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

The methods described herein are carried out by administering to the subject an effective amount of an agent that reduces Arg2 transcription or activity, thereby treating or preventing endothelial dysfunction or an endothelial dysfunction-associated condition in the subject. Preferably, endothelial nitric oxide (NO) production is increased.

Endothelial dysfunction-associated conditions suitable for treatment with the methods described herein include atherosclerosis, chronic obstructive pulmonary disease (COPD), vasculitis (e.g., systemic, pulmonary, and cerebral vasculitis), hypertension (e.g., pulmonary and systemic hypertension), and local and systemic inflammatory responses during infectious, autoimmune, diabetes-induced vascular dysfunction, and systemic sepsis-like syndromes.

An exemplary agent that reduces Arg2 transcription or activity comprises HDAC2 or an HDAC2 agonist. For example, the HDAC2 comprises an HDAC2 polynucleotide or HDAC2 polypeptide. In some cases, the HDAC2 polynucleotide comprises HDAC2 complementary deoxyribonucleic acid (cDNA) or HDAC2 messenger ribonucleic acid (mRNA). In some cases, the HDAC2 polynucleotide is administered via a vector, e.g., an adenoviral vector. The HDAC2 reagent or agonist is intended to increase HDAC2 signaling in the target cells.

For example, the HDAC agonist comprises a small molecule agonist of HDAC2. An exemplary small molecule agonist of HDAC2 comprises a methylated xanthine (methylxanthine) and curcumin.

A small molecule is a compound that is less than 2000 daltons in mass. Typically, small molecules are less than one kilodalton. The molecular mass of the small molecule is preferably less than 1000 daltons, more preferably less than 600 daltons, e.g., the compound is less than 500 daltons, 400 daltons, 300 daltons, 200 daltons, or 100 daltons. Small molecules are organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecule inhibitors can be synthetically engineered to consist of a fragment, or small portion, or a longer amino acid chain to fill a binding pocket of an enzyme.

In high doses methylxanthine can lead to convulsions that are resistant to anticonvulsants. As such, the methylxanthine is administered at a low dose of 1 mg/kg/day-1 g/kg/day, e.g., about 1 mg/kg/day, about 2 mg/kg/day, about 3 mg/kg/day, about 4 mg/kg/day, about 5 mg/kg/day, about 6 mg/kg/day, about 7 mg/kg/day, about 8 mg/kg/day, about 9 mg/kg/day, about 10 mg/kg/day, about 15 mg/kg/day, about 20 mg/kg/day, about 25 mg/kg/day, about 50 mg/kg/day, about 75 mg/kg/day, about 100 mg/kg/day, about 150 mg/kg/day, about 200 mg/kg/day, about 250 mg/kg/day, about 300 mg/kg/day, about 350 mg/kg/day, about 400 mg/kg/day, about 450 mg/kg/day, about 500 mg/kg/day, about 550 mg/kg/day, about 600 mg/kg/day, about 650 mg/kg/day, about 700 mg/kg/day, about 750 mg/kg/day, about 800 mg/kg/day, about 850 mg/kg/day, about 900 mg/kg/day, about 950 mg/kg/day, or about 1 g/kg/day.

Suitable methylxanthines comprises caffeine, aminophylline, 3-isobutyl-1-methylxanthine (IBMX), paraxanthine, pentoxifylline, theobromine, and theophylline (1,3-dimethylxanthine).

In some cases, a composition of the invention is administered orally or systemically. Other modes of administration include rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

Pharmaceutical compositions may be assembled into kits or pharmaceutical systems for use in treating a condition associated with endothelial dysfunction. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the kit.

Described herein are methods of treating or preventing endothelial dysfunction or an endothelial dysfunction-associated condition in a subject, the method comprising: identifying a subject having or at risk of developing endothelial dysfunction or an endothelial dysfunction-associated condition and administering to the subject an effective amount of an agent that reduces NEDDylation activating enzyme (NAE) activity, thereby treating or preventing endothelial dysfunction or an endothelial dysfunction-associated condition in the subject. An endothelial dysfunction-associated condition suitable for treatment with an agent that reduces NEDDylation activating enzyme (NAE) activity is atherogenesis.

Agents that reduce NEDDylation activating enzyme activity comprise an HDAC agonist, a HDAC, a NAE inhibitor, and/or any combination thereof. For example, the NAE inhibitor comprises MLN4924. In some cases, the HDAC comprises an HDAC polynucleotide or HDAC polypeptide. In some cases, the HDAC polynucleotide comprises HDAC complementary deoxyribonucleic acid (cDNA) or HDAC messenger ribonucleic acid (mRNA). In a preferred embodiment, the HDAC comprises HDAC2. In some cases, the HDAC agonist comprises a small molecule agonist of HDAC2.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “binding to” a molecule is meant having a physicochemical affinity for that molecule.

“Detect” refers to identifying the presence, absence or amount of the analyte to be detected.

By “disease” is meant any condition or disorder that damages or interferes with the normal function of a cell, tissue, or organ. Examples of diseases include those associated with endothelial dysfunction.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the invention also comprises polypeptides and nucleic acid fragments, so long as they exhibit the desired biological activity of the full length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “NEDDylation” is meant the process by which the ubiquitin-like protein NEDD8 is conjugated to its target proteins. NEDD8 (neural-precursor-cell-expressed developmentally down-regulated 8) is a protein involved in the regulation of cell development, growth, and viability. During NEDDylation, NEDD8 links itself to a protein through an isopeptide linkage between its carboxy-terminal glycine and the lysine of the substrate. NEDDylation of the substrate causes a structural change with three results: 1) a conformational change in the substrate which may restrict molecular movement and the positioning of binding partners, 2) it incompatibility of the target protein with binding partners, and 3) recruiting NEDD8-interacting proteins. When NEDD8 binds to the ubiquitin E2 Ubc4, the interaction stimulates cullin-based ubiquitin ligases. NEDDylation relies on its own E1 and E2 enzymes despite any similarities to ubiquitination.

By “reduces” is meant a negative alteration of at least 5%, 10%, 25%, 50%, 75%, or 100%.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1A-FIG. 1D is a series of line graphs showing that histone deacetylase (HDAC) inhibition by trichostatin A (TSA) impairs vascular relaxation that involves arginase 2 (Arg2). FIG. 1A and FIG. 1B are line graphs showing the dose-response effect of acetylcholine (FIG. 1A) and sodium nitroprusside (SNP; FIG. 1B) on vascular relaxation, which was determined using wire myography in the presence of the HDAC inhibitor, TSA (200 nmol/L) either alone or when coincubated with the arginase inhibitor amino-2-borono-6-hexanoic acid (ABH; 1 mmol/L). FIG. 1C and FIG. 1D are line graphs showing the dose response of acetylcholine (FIG. 1C) and SNP-mediated relaxation (FIG. 1D) as determined in Arg2 knockout mice (Arg2−/−) in the presence or absence of TSA (200 nmol/L). *P<0.05 vs control; n=6. There was no difference in the absolute tension generated with phenylephrine exposure by aortic segments from each of the experimental groups. ns indicates not significant; and WT, wild type.

FIG. 2A-FIG. 2J is a series of bar charts and photographs of Western blots (WB) demonstrating HDAC inhibition upregulates Arg2 activity, messenger RNA (mRNA), and protein expression. FIG. 2A is a bar chart showing arginase activity in human aortic endothelial cells (HAECs) after incubation with 0.4 μmol/L of TSA for 18 hours. Arginase activity was measured in cell lysates using a urea production assay. FIG. 2B is a photograph of a WB showing Arg2 protein levels. FIG. 2C and FIG. 2D are photographs of Western blots showing the dose-dependent (0, 0.1, 0.3, and 1 μmol/L; FIG. 2C) and time-dependent (0, 2, 4, 8, and 18 hours; FIG. 2D) effects of TSA (1 μmol/L) on endothelial Arg2 protein expression. FIG. 2E is a photograph of a Western blot showing arginase activity. HAECs were transduced with 15 multiplicity of infection (MOI) of adenoviruses encoding either nontargeted, Arg2 or Arg1 short hairpin RNA (shRNA). The effect of TSA on total arginase activity was determined using the urea assay. FIG. 2F is a photograph and FIG. 2G is a bar chart showing the effect of TSA (1 μmol/L) on Arg2 mRNA levels as determined using reverse transcriptase polymerase chain reaction (RT-PCR; FIG. 2F) and real-time PCR (FIG. 2G) in HAECs with increasing doses of TSA (0, 0.1, 0.2, and 1 μmol/L) or nicotinamide 0.1 mmol/L or 0.2 mmol/L (FIG. 2F only, lanes 4 and 5). FIG. 2H is a bar chart showing mRNA levels as determined by real-time PCR in isolated mice aortas that were incubated with 200 nmol/L TSA for 18 hours. FIG. 2I is a bar chart showing arginase activity as determined from isolated segments of mice aorta exposed to TSA (200 nmol/L). FIG. 2J is a bar chart showing arginase activity as determined from HAECs exposed to TSA (1 μmol/L) or nicotinamide (5 mmol/L) for 30 minutes. *P<0.05 vs control (no TSA), ^{$}P<0.05 vs control (no TSA), ^{#}P<0.05 vs control (with TSA). WB and RT-PCR experiments were performed 3 to 4 times. Fold change in real-time PCR data indicates the differences in the ratio between Arg2 mRNA and 18s mRNA when the TSA-treated and control sample data are measured. IB indicates immunoblot; and RLU, relative light units.

FIG. 3 is a photograph of an immunoblot showing that trichostatin A does not affect Arg2 ubiquitination. Human aortic endothelial cells coexpressing FLAG epitope-tagged Arg2 and HA-tagged ubiquitin were subjected to immunoprecipitation by FLAG antibody and immunoblotted for HA using anti-HA antibody. All groups were treated with the proteasomal inhibitor MG132 (10 μmol/L) for the last 8 hours before IP. Results are representatives of 3 independent experiments. IB indicates immunoblot.

FIG. 4A-FIG. 4C is a series of a photograph of a Western blot and bar charts showing the effects of TSA on arginase transcription and promoter activity. FIG. 4A is a photograph of a Western blot wherein HAECs were pretreated with either 10 μg/mL of the transcriptional inhibitor actinomycin D (Act D) or 10 μg/mL of the protein translation inhibitor cycloheximide for 8 hours. Cell lysates were then subjected to Western blot (WB) using antibodies against Arg2 and Glyceraldehyde 3-phosphate dehydrogenase (GAPDH). FIG. 4B is a bar chart showing luciferase activity as determined in HAECs that were transfected with the pGL3 luciferase (LUC) reporter plasmid consisting of the −1 kb human ARG2 promoter and 3 different promoter fragments (as illustrated above the graph) and incubated in the presence of TSA. FIG. 4C is a bar chart showing luciferase activity, wherein HAECs were transfected with pgL3.4 harboring the 1-kb Arg2 promoter and challenged with increasing doses of TSA. *P<0.05 vs control, and WB results are representatives of 3 independent experiments. IB indicates immunoblot; and RLU, relative light units.

FIG. 5A-FIG. 5D is a series of photographs and bar charts showing that attenuated nitric oxide production in TSA-treated HAECs was restored by arginase inhibition. HAECs were incubated with either TSA (1 μmol/L) alone or together with amino-2-borono-6-hexanoic acid (ABH; 1 mmol/L). FIG. 5A shows representative epifluorescence images, while FIG. 5B is a bar chart demonstrating quantitative analysis of DAF-DA fluorescence, both of which were used to assess nitric oxide (NO) production. FIG. 5C is a bar chart showing byproducts of NO, mainly nitrite, in HAEC cell media that were incubated with either TSA alone or coincubated with TSA and ABH. A NO analyzer from Siever was used to measure byproducts of NO. FIG. 5D is a bar chart showing reactive nitrogen species (RNS) levels. Human embryonic kidney 293 cells that do not express native endothelial nitric oxide synthase (eNOS) were transduced with constitutively active eNOS constructs and incubated with either TSA alone or together with ABH. The dichloro-dihydro-fluorescein diacetate (DCFH-DiOxyQ)-based fluorescent assay from Cell Biolabs was used to determine NO radicals in cell culture media. *P<0.05 vs control, #P<0.05 vs TSA alone, $P<0.05 vs TSA+ABH. DAF fluorescence images are representative of 3 independent experiments. CIeNOS indicates calcium-insensitive endothelial nitric oxide synthase; RFU, relative fluorescence units; and RNS, reactive nitrogen species.

FIG. 6A-FIG. 6H is a series of phogographs, a bar chart, and line graphs showing that class I HDACs regulate Arg2 expression and vascular function. FIG. 6A-FIG. 6C is a series of photographs of immunoblots showing Arg2 expression as measured by immunoblotting using anti-Arg2 polyclonal antibody to assay human aortic endothelial cell lysates that had been incubated with several commercially available class-specific HDAC inhibitors (EC50 in Table I). FIG. 6D is a bar chart showing a graphical representation of the results presented in FIG. 6A-FIG. 6C. FIG. 6E is a line graph showing the dose-dependent effects of acetylcholine on vessel relaxation. FIG. 6F is a line graph showing the dose-dependent effects of SNP on vessel relaxation. Isolated aortas from C57/BL6 mice were incubated in EBM2 media containing 1 μmol/L of MGCD0103 for 18 hours at 37° C. and dose-dependent effects of acetylcholine (FIG. 6E) and SNP (FIG. 6F) on vessel relaxation was determined using wire myography. FIG. 6G is a line graph showing a dose response curve of acetylcholine-mediated relaxation, and FIG. 6H is a line graph showing a dose reponse curve of SNP-mediated relaxation as determined in aortic rings from Arg2 knockout mice (Arg2−/−) that were incubated with either vehicle alone or with MGCD0103 (1 μmol/L) for 18 hours. *P<0.05 vs control; n=6. Western blot results are representative of 3 to 4 independent experiments. eNOS indicates endothelial nitric oxide synthase; IB, immunoblot; ns, not significant; TSA, trichostatin A; and WT, wild type.

FIG. 7A-FIG. 7E is a series of photographs of immunoblots showing that hHDAC2 is the specific HDAC isoform that regulates Arg2 expression. Human aortic endothelial cells were transfected with increasing concentrations of HDAC1 (FIG. 7A), HDAC3 (FIG. 7B), HDAC8 (FIG. 7C), or HDAC2 (FIG. 7D) cDNAs, and Arg2 expression was determined by Western blot. FIG. 7E is a photograph of an immunoblot wherein HAECs that were transfected with HDAC2 siRNA (30 nmol/L) were subjected to immunoblotting for Arg2 and HDAC2. Results are representatives of 3 to 4 independent experiments.

FIG. 8A-FIG. 8F is a series of bar charts and photographs of immunoblots showing that increased endothelial Arg2 expression in response to oxidized low-density lipoprotein (OxLDL) involves promoter activation and HDAC2. FIG. 8A is a bar chart and photograph of an immunoblot showing Arg2 and GAPDH protein levels in HAECs after exposure to OxLDL. HAECs were exposed to 50 μg/mL of OxLDL for 18 hours and subjected to Western blot (WB) using Arg2 antibody. FIG. 8B is a bar chart showing luciferase activity as determined in HAECs that were transfected with pGL3 luciferase (LUC) reporter plasmids consisting of either −1 kb human ARG2 promoter or 1 of 3 different promoter fragments (as illustrated above the graph in FIG. 4B) and incubated with 50 μg/mL of OxLDL for 18 hours. FIG. 8C is a bar chart wherein HAECs were transfected with an LUC reporter plasmid containing the −1-kb human ARG2 promoter, and cells were exposed to increasing doses of OxLDL (0, 25, 50, and 100 μg/mL) for 18 hours. Firefly luciferase activity was measured and normalized to Renilla luciferase. GFP and HDAC2 adenoviruses were added 8 hours after transfection with the luciferase constructs. FIG. 8D is a shematic and a photograph of an immunoblot showing the binding of HDAC2 to the Arg2 promoter, as quantified using a chromatin immunoprecipitation (ChIP) assay. FIG. 8E is a photograph of an immunoblot, wherein HAECs were exposed to increasing doses of OxLDL (0, 25, 50, and 100 μg/mL) for 18 hours and immunoblotted for HDAC2 and Arg2. FIG. 8F is a photograph of an immunoblot, wherein HAECs with adenoviral-mediated HDAC2 overexpression or controls (Ad-GFP) were exposed to OxLDL, and expression levels of Arg2, HDAC2, and GAPDH (loading control) were determined with WB. *P<0.05 vs control. WB results are representatives of 3 to 4 independent experiments. IB indicates immunoblotting; and RLU, relative light units.

FIG. 9A-FIG. 9E is a series of line graphs photographs of immunoblots, and bar charts showing that OxLDL-mediated impairment of vascular relaxation is ameliorated by HDAC2 overexpression. Isolated mice aortic rings were incubated in EBM2 media containing 100 MOI of either GFP or HDAC2 adenoviruses. Twenty four hours post transduction, media was replaced with fresh media with or without OxLDL (50 μg/mL) for 48 hours. FIG. 9A is a line graph showing the dose-response effects of acetylcholine on vascular relaxation as determined using wire myography. FIG. 9B is a line graph showing the dose-response effects of SNP on vascular relaxation as determined using wire myography. FIG. 9C is a series of photographs of immunoblots showing green fluorescent protein (GFP) and HDAC2 expression. Mice aortic segments were transduced with 100 MOI of either GFP or HDAC2 adenoviruses and subjected to immunoblotting with anti-GFP and anti-HDAC2 antibodies. FIG. 9D is a bar chart and a series of photographs of immunoblots, wherein HAECs overexpressing HDAC2 or GFP were subjected to an arginase activity assay. FIG. 9E is a bar chart showing nitric oxide production as determined using the DAF-FM DA fluorescence assay in HAECs that were transduced with either Ad-Arg2shRNA or Ad-HDAC2shRNA alone, or both reagents in combination. Nontargeted Ad-shRNA was used as a control. *P<0.05 vs control; n=6. RFU indicates relative fluorescence units; and WT, wild type.

FIG. 10A-FIG. 10B is a series of bar charts showing densitometric analysis of Arg2 expression. FIG. 10A is a bar chart showing dose-dependent effects of TSA on Arg2 expression as quantified using densitometry and normalized to GAPDH expression. FIG. 10B is a bar chart showing time-dependent effects of TSA on Arg2 expression as quantified using densitometry and normalized to GAPDH expression.

FIG. 11A-FIG. 11B is a series of photographs of blots demonstrating that HDAC8 is expressed in HAEC, but does not regulate Arg2 expression. FIG. 11A is a photograph of a blot showing the expression of HDAC7 and HDAC8. Total RNA was extracted from using trizol reagent followed by RNA purification. cDNA was synthesized using total RNA as a template and using oligodT and reverse transcriptase. Primers specific to HDAC7 and HDAC8 were used to detect the expression of HDAC7 and HDAC8. FIG. 11B is a photograph of a blot showing HDAC8 and Arg2 expression levels. Increasing dose of HDAC8 siRNA (0, 5, 10 and 15 nM) was transfected in HAEC and post 72 hours, cell lysates were subjected to immunoblotting with HDAC8 and Arg2 antibody.

FIG. 12A-FIG. 12B is a series of photographs of blots showing that HDAC4-7 do not regulate Arg2 expression. FIG. 12A is a photograph of blots showing Arg2, FLAG and GAPDH expression, wherein FLAG-tagged cDNA encoding HDAC4-7 in pcDNA3.1 was expressed in HAEC and cell lysates were subjected to immunoblotting using Arg2, FLAG and GAPDH antibody. FIG. 12B is a photograph of blots showing Arg2, FLAG and GAPDH expression, wherein FLAG-tagged HDAC7 was expressed in HAEC in increasing concentration (0, 100, 300 and 1000 ng) and cell lysates were subjected to immunoblotting using Arg2, FLAG and GAPDH antibodies.

FIG. 13 is a series of photographs of blots showing that increased global cell acetylation with calcium-binding protein (CBP) does not influence Arg2 expression. Increasing concentrations of high affinity CBP (HA-CBP; 0, 300 ng and 1 μg) were transiently expressed in HAEC, and cell lysates were subjected to immunoblotting for Arg2, HA, and GAPDH.

FIG. 14A-FIG. 14E is a series of bar charts showing densitometry analysis of HDAC2 and Arg2 expression. FIGS. 14A, 14B, 14C, 14D, and 14E are bar charts showing the results from immunoblots from FIGS. 7D, 7E, 8E, 8F and 9C, respectively, after being subjected to densitometry analysis of Arg2 and HDAC2 expression. Each value was normalized to GAPDH expression for the respective sample.

FIG. 15A-FIG. 15B is a series of photographs of immunoblots showing validation of Adenoviral HDAC2 and Arg2 shRNA in HAEC. HAEC were transduced with non-targeted shRNA (30MOI), HDAC2 shRNA (5, 10 and 30 MOI), and Arg2 shRNA (30MOI), and lysates were subjected to immunoblotting with antibodies against HDAC2 or Arg2, respectively, and with GAPDH antibodies.

FIG. 16A-FIG. 16B is a series of graphs showing that augmented histone deacetylase 2 (HDAC2) expression provides protection against oxidized low-density lipoprotein (OxLDL)-mediated endothelial dysfunction. FIG. 16A shows isolated mice aortic rings were transduced with either GFP (green fluorescent protein) or HDAC2 adenoviruses (100 multiplicity of infection (MOI)) for 24 hrs and incubated for an additional 24 hrs in the presence or absence of OxLDL (50 μg/mL). After 48 hours, dose-response effects of Ach (acetylcholine) on vascular relaxation were determined using wire myography. FIG. 16B shows NO (nitric oxide) production as determined using the DAF-FM DA (4-Amino-5-Methylamino-2′,7′-Difluorofluorescein Diacetate) fluorescence assay in HAEC (Human aortic endothelial cells) that were transduced with either Ad-Arg2shRNA (adenoviral Arg2 small (or short) hairpin RNA) or Ad-HDAC2shRNA (adenoviral histone deacetylase 2 small (or short) hairpin RNA) alone, or with both reagents in combination.

FIG. 17A-FIG. 17C is a series of graphs showing that in vivo HDAC2 gene transfer improves vascular endothelial function in ApoE KO mice fed a high fat diet. FIG. 17A shows total RNA was extracted from intact aortas isolated from C57BL6 (WT) or ApoE−/− mice fed an atherogenic diet. cDNA was synthesized followed by semiquantitative RT PCR using taq polymerase with HDAC2- and GAPDH-specific primers. FIG. 17B shows dose-response to Ach in aortic rings isolated from ApoE−/− mice with an atherogenic diet that were subjected to 3 injections of either adenoviral HDAC2 or GFP (control). FIG. 17C shows HDAC2 expression in isolated aortas from FIG. 17B.

FIG. 18A-FIG. 18E is a series of photographs and graphs showing generation and characterization of hHDAC2 transgenic mice. FIG. 18A and FIG. 18B shows primers specific to human HDAC2 were used for PCR analysis using Genomic DNA isolated from the tails of the following mice as templates: for FIG. 18A C57BL6 (lane 2), non-transgenic littermates (lane 3), and an HDAC2 transgenic founder (lane 4); a transgenic DNA construct that was injected into the murine ooytes was used as positive control template (lane 1); for FIG. 18B F3 positive control; G3 through E4, HDAC2 positive pups exhibiting high (C4), medium (A4), and low (E4) expression levels. FIG. 18C and FIG. 18D shows snap frozen isolated aortas from WT and HDAC2-Tg mice (FIG. 18C), and Aortas from WT and endothelium-denuded (E−) aortas from HDAC2-Tg mice (FIG. 18D) were subjected to western blotting using anti-HDAC2, eNOS and GAPDH (loading control) antibodies. FIG. 18E shows bone marrow was harvested from femurs, and subjected to western blotting using anti-HDAC2 and GAPDH antibodies-bone marrow precursors did not exhibit increased HDAC2 expression and this demonstrates that expression of the transgenic human HDAC2 was restricted to vascular endothelium.

FIG. 19A-FIG. 19C is a series of graphs and photographs of immunoblots showing transgenic mice with endothelial-specific HDAC2 overexpression (HDAC2-Tg) exhibit enhanced endothelial NO production and endothelial-dependent vasorelaxation. FIG. 19A shows isolated aortic rings from WT and HDAC2-Tg mice were incubated with OxLDL (50 μg/mL) for 48 hours and dose-response to Ach was determined. FIG. 19B shows isolated aortas from WT or HDAC2-tg mice were loaded with DAF2 FM for NO measurement. FIG. 19C shows C57BL6 mice were injected with adeno-associated viruses encoding murine PCSK9 D377Y cDNA with a liver-specific promoter. Liver lysates were immunoblotted for PCSK9& LDL-r.

FIG. 20 is a diagram showing a schematic for a proposed pathway for HDAC2 modulation of endothelial Arg2 and resulting endothelial dysfunction. OxLDL downregulates HDAC2 expression via NEDD8- and/or Ubiquitin-mediated proteasomal degradation. A drop in HDAC2 levels leads to transcriptional upregulation of Arg2, L-arginine depletion, eNOS uncoupling, and EC dysfunction.

FIG. 21A-FIG. 21H is a series of graphs and photographs of immunoblots showing inhibition of NEDDylation by MLN4924 improves OxLDL-mediated vascular endothelial dysfunction via downregulation of Arginase 2. Wire myography was used to measure vasorelaxation with acetylcholine (FIG. 21A, Ach) and sodium nitroprusside (FIG. 21B, SNP) in isolated mice aortas. Conditions included 24 h of OxLDL (50 μg/mL) or control medium preceded by 1 h with either vehicle (DMSO) or MLN4924 (1 μM). FIG. 21C shows HAEC were exposed to OxLDL (50 μg/mL) for 24 h followed by 8 h of MG132 (10 μM) and lysates were immunoblotted for NEDD8 and GAPDH. FIG. 21D and FIG. 21E shows HAEC were preincubated with DMSO, MG132 (10 μM), or MLN4924 (1 μM) for 8 h followed by stimulation with OxLDL (50 μg/mL) for 24 h and lysates were immunoblotted for Arg2, HDAC2 and GAPDH, or assayed for arginase activity. Note that native (and not transgene) HDAC2 protein levels are shown here. FIG. 21F shows HAEC were incubated with DMSO orMLN4924 (0, 0.1, 0.3 or 1 μM) and lysates were immunoblotted for HDAC2 and GAPDH. FIG. 21G shows HAEC were incubated with either vehicle (DMSO) orMLN4924 (1 μM) and arginase activity was measured in lysates. FIG. 21H shows HAEC expressing HA-epitope-tagged NEDD8 were incubated with DMSO or MLN4924 (0, 0.1, 0.3 or 1 μM) and lysates were immunoblotted with HA, HDAC2 and GAPDH antibodies. Note that “0 μgHA-NEDD8” denotes an empty vector (pcDNA3.1) control. *Indicates p b 0.05 vs control; **indicates p b 0.001; n=6 for vascular reactivity and arginase activity assays.

FIG. 22A-FIG. 22I shows NEDDylation regulates HDAC2 turnover in vascular endothelium. FIG. 22A shows 24 h after incubation of HAEC with OxLDL (50 μg/mL)(or control), cells were treated with cycloheximide (10 μg/mL) for 0, 2, 4, 6 or 8 h, and cell lysates were subjected to immunoblotting with anti-HDAC2 and GAPDH antibodies. FIG. 22B shows a graph of cycloheximide chase data from FIG. 22A. FIG. 22C shows isolated mouse aortas with intact (E+) or denuded endothelium (E−) were exposed to OxLDL for 24 h and tissue homogenates were subjected to immunoblotting with HDAC2 and GAPDH antibodies. FIG. 22D shows HAEC were transfected with FLAG-tagged HDAC2 and HA-tagged NEDD8; after 48 h, they were incubated with MG132 (10 μM) for 8 h and cell lysates were subjected to immunoprecipitation using either IgG (control) or anti-FLAG antibodies and immunoblotted with anti-HA antibody. FIG. 22E shows HAEC were incubated with OxLDL (50 μg/mL) or control for 24 h, then 6 h with MG132 (10 μM); lysates were subjected to immunoprecipitation with anti-IgG (control) or anti-HDAC2 antibodies and immunoblotted with anti-NEDD8 and anti-HDAC2 antibodies (middle and bottom panels, respectively). Densitometry analysis of data from three experiments is shown in top panel. FIG. 22F shows HAEC were transfected with HA-NEDD8 cDNA (0, 0.1, 0.3 or 1 μg) and subjected to immunoblotting after 48 h with anti-HA and anti-GAPDH antibodies. Note that “0 μg HA-NEDD8” denotes an empty vector (pcDNA3.1) control. FIG. 22G shows HAEC were transfected with HA-NEDD8 cDNA (0, 0.1, 0.3 or 1 μg) and after 48 h cells were incubated with MG132 (10 μM) for 8 h; lysates were immunoblotted with anti-HA, HDAC2 and GAPDH antibodies. FIG. 22H shows a graph of densitometric analysis of data from three experiments for FIG. 22F and FIG. 22G (*denotes p <0.05 0.3 μg NEDD8 transfected group without MG132 treated vs MG132 treated; #denotes p <0.05 1 μg NEDD8 transfected group without MG132 treated vs MG132 treated). FIG. 22I shows HAEC were transfected with SENP8 cDNA (0, 0.1, 0.3 or 1 μg), and lysates were immunoblotted with anti-HDAC2 and GAPDH antibodies. Western blots in this figure are representative of 3 independent experiments.

FIG. 23A-FIG. 23C is a series of graphs and photographs of immunoblots depicting HDAC2 expression. HDAC2 expression has an impact on phosphorylation of AKT on serine 473 and threonine 308 in HAEC. HAEC were transduced with 30 MOI of adenoviruses encoding either 1) GFP (Control), 2) HDAC2 cDNA 3) Non targeted shRNA or 4)HDAC2 shRNA. Post 48 hours, cells lysates were subjected to western-blotting with HDAC2, phospho AKT (Serine 473 and Threonine 308) and total AKT antibodies.

FIG. 24A-FIG. 24B is a series of graphs showing systolic blood pressure and pulse wave velocity in transgenic and wild type mice. Transgenic HDAC2-Tg mice exhibit lower systolic blood pressure and pulse wave velocity (a measure of aortic stiffness) than their WT counterparts at 10 weeks of age. Pulse Wave Velocity (PWV) and blood pressure (BP) were measured in real time in anesthesized WT or HDAC2-Tg mice using a high frequency Doppler (Indus Instruments) and a Scisense dual high fidelity pressure catheter as previously described ((Steppan J, et al. 2014 J. Am. Heart Assn., 3(2):e000599)(Kuo M M, et al. 2014 JoVE.(94))(Jung S M, et al. 2013 Am J Physiol Heart Circ Physiol., 305(6):H803-810)). HDAC2-Tg mice were generated using the construct scheme depicted in FIG. 28.

FIG. 25 is a dose response curve showing that aortic strips from HDAC2-Tg mice exhibit a substantially greater vasorelaxation response to acetylcholine than aortic samples from WT mice. Aortic strips from HDAC2-Tg mice exhibit a substantially greater vasorelaxation response to acetylcholine than aortic samples from WT mice. In vivo HDAC2 overexpression provides protection against high fat diet induced endothelial dysfunciton. PCSCK9 gain of function mutant AAV injected HDAC2-Tg and C57BL6 mice (8 weeks old) were subjected to high fat/high cholesterol diet regimen for 16 weeks and endothelial dependent and independent relaxation were assessed in isolated aortic rings from these mice supplemented with increasing dose of A) acetylcholine or B) Sodium Nitroprusside respectively by wire myography. C) eNOS dependent component was determined by incubating rings in 10 uM of LNAME.

FIG. 26A-FIG. 26C is series of graphs and photographs of immunoblots showing PCSK9 in HDAC2-Tg and WT mice. FIG. 26A shows HDAC2-Tg and WT mice were transduced with a gain-of-function mutation of PCSK9 using an adeno-associate viral vector in order to rapidly induce atherogenesis as per the model of Bjorklund et al. (Circ Res. 2014; 114). FIG. 26B shows levels of the LDL receptor were successfully reduced to a minimal presence. FIG. 26C shows that despite this reduction in the LDL receptor, HDAC2-Tg mice exhibited an approximately 10-fold rate of non-HDL cholesterol clearance as compared with WT mice that were also transduced with PCSK9.

FIG. 27A-FIG. 27B is a graph and photograph of an immunoblot showing in vivo HDAC2 transduction. FIG. 27A shows HDAC2 expression in isolated aortas from ApoE−/− mice that were fed an atherogenic diet following 3 injections of either adenoviral GFP (control) or HDAC2 or GFP (control). FIG. 27B shows vasorelaxation dose-response to acetylcholine in aortic rings isolated from ApoE−/− mice that were fed an atherogenic diet following 3 injections of either adenoviral GFP (control) or HDAC2 or GFP (control).

FIG. 28 shows a schematic depicting the design strategy for the human HDAC2 construct that was used to make the transgenic mouse line with human HDAC2 overexpression that is specific to the vascular endothelium. The transgenic mice resulting from this construct were used in numerous experiments, as demonstrated in FIG. 18C, FIG. 19B and FIG. 24.

FIG. 29A-FIG. 29B is a series of graphs showing dose-dependent vasorelaxation of isolated aortic rings with or without pretreatment with MGCD0103 (0.41M), MLN4924(1.0 μM), or both (n=4-10 per group). FIG. 29A shows that vascular endothelial function was tested by acetylcholine (Ach) dose response via force-tension myography. Preincubation with MGCD0103 for 48 hours led to a significant attenuation of Ach response in WT aorta while MLN4924 did not cause any significant changes. MLN4924 did not reverse the vascular endothelial dysfunction induced by MGCD0103. FIG. 29B shows that measurements of endothelium-independent responses to SNP demonstrated no significant differences in dose-dependent vasorelaxation in any of the four groups that were studied. * denotes p<0.05; NS indicates p >0.05.

FIG. 30 is an immunoblot showing that HAEC expressing FLAG-tagged HDAC2 alone was subjected to immunoprecipitation with either IgG (isotype control) or FLAG antibody and immunoblotted with anti-HA antibody. Arrow indicates Ig heavy chain.

### DETAILED DESCRIPTION

The invention is based, at least in part, on the surprising discovery that (1) HDAC2 holds a major vascular injury mechanism in check (namely endothelial Arginase 2); and (2) oxidative injury to vascular endothelium enhances the degradation of HDAC2 by the degradative process known as neddylation. Specifically, HDAC2 tonically limits the availability of the eNOS competitor Arg2 in vascular endothelium by constraining Arg2 transcription in baseline homeostatic conditions, and that this brake on Arg2 population in HAECs is released by oxidative injury. Described herein are therapeutic interventions that restore and preserve vascular endothelial health against the epidemic onslaught of oxidative injury and atherogenesis.

### Atherosclerosis

Atherosclerotic cardiovascular disease is the most important cause of mortality in the Western world. Its pathobiology involves chronic inflammation of the vascular wall, resulting from endothelial dysfunction, adhesion molecule expression, and monocyte infiltration of the intima, ultimately leading to plaque development.

Atherosclerosis (also known as arteriosclerotic vascular disease or ASVD) is a specific form of arteriosclerosis in which an artery wall thickens as a result of invasion and accumulation of white blood cells (WBCs). The accumulation of the WBCs is termed “fatty streaks” early on because of appearance being similar to that of marbled streak. These accumulations contain both living, active, WBCs (producing inflammation) and remnants of dead cells, including cholesterol and triglycerides. The remnants eventually include calcium and other crystallized materials, within the outer-most and oldest plaque. The “fatty streaks” reduce the elasticity of the artery walls. However, they do not affect blood flow for decades, because the artery muscular wall enlarges at the locations of plaque. The wall stiffening may eventually increase pulse pressure; widened pulse pressure is one possible result of advanced disease within the major arteries

Atherosclerosis is therefore a syndrome affecting arterial blood vessels due to a chronic inflammatory response of WBCs in the walls of arteries. This is promoted by low-density lipoproteins (LDL, plasma proteins that carry cholesterol and triglycerides) without adequate removal of fats and cholesterol from the macrophages by functional high-density lipoproteins (HDL). It is commonly referred to as a “hardening” or furring of the arteries. It is caused by the formation of multiple atheromatous plaques within the arteries. Atherosclerosis can lead to serious problems, including heart attack, stroke, or even death.

Areas of severe narrowing, stenosis, detectable by angiography, and to a lesser extent “stress testing” have long been the focus of human diagnostic techniques for cardiovascular disease, in general. However, these methods focus on detecting only severe narrowing, not the underlying atherosclerosis disease. As such, other diagnostic methods have been developed for earlier detection of atherosclerotic disease. Some of the detection approaches include anatomical detection and physiologic measurement. Examples of anatomical detection methods include (1) coronary calcium scoring by CT, (2) carotid IMT (intimal media thickness) measurement by ultrasound, and (3) intravascular ultrasound (IVUS). Examples of physiologic measurement methods include (1) lipoprotein subclass analysis, (2) HbA1c, (3) hs-CRP, and (4) homocysteine.

Both anatomic and physiologic methods allow early detection before symptoms show up, disease staging and tracking of disease progression. Anatomic methods are more expensive and some of them are invasive in nature, such as IVUS. On the other hand, physiologic methods are often less expensive and safer; however, they do not quantify the current state of the disease or directly track progression. In the recent years, ways of estimating the severity of atherosclerotic plaques has been made possible with the developments in nuclear imaging techniques such as positron emission tomography (PET) and single-photon emission computed tomography (SPECT).

Atherosclerosis can affect any artery in the body, including arteries in the heart, brain, arms, legs, pelvis, and kidneys. As a result, different diseases may develop based dependent upon which arteries are affected. Artherosclerotic diseases include coronary heart disease, carotid artery disease, peripheral arterial disease, and chronic kidney disease.

### Regulation of Gene Expression in Endothelial Dysfunction

It is well established that oxidized low-density lipoprotein (OxLDL) is one of the most important proatherosclerotic molecules and its effects are mediated by binding to the lectin-like OxLDL receptor (LOX-1) and thence by stimulation of proinflammatory gene expression, reactive oxygen species production, and downregulation of endothelial protective nitric oxide (NO) production (Mitra et al., 2011 Cardiovasc Drugs Ther, 25:419-429; and Sawamura et al., 2012 Curr Opin Lipidol, 23:439-445). It has previously been demonstrated that exposure of endothelium to OxLDL induces the activation of arginase 2 (Arg2), resulting endothelial nitric oxide synthase (eNOS) uncoupling because of substrate 1-arginine depletion. This in turn leads to an increase in eNOS-dependent ROS generation and a decrease in NO production (Sawamura et al., 2012 Curr Opin Lipidol, 23:439-445; Ryoo et al., 2011 Atherosclerosis, 214:279-287; and Ryoo et al., 2008 Circ Res, 102:923-932). Furthermore, it has been demonstrated that both biochemical inhibition and genetic knockdown of endothelial Arg2 prevent eNOS uncoupling, endothelial dysfunction, and atherosclerotic plaque burden in atherogenic mice (Ryoo et al., 2008 Circ Res, 102:923-932).

As described herein, the data suggest that the increase in endothelial Arg2 activity is dependent on 2 events, 1 of which is early and another that occurs later and is more long-lasting. The early process involves a posttranslational event: subcellular decompartmentalization from mitochondria where it resides in quiescent cells (Pandey et al., 2014 Circ. Res., 115(4):450-9; Lim et al., 2007 Am J Physiol Heart Circ Physiol, 293:H3317-H3324) to the cytoplasm. The later regulatory process involves a transcriptional event that leads to an upregulation in Arg2 gene expression (Pandey et al., 2014 ATVB, 34(7):1556-1566). Given the critical role of Arg2 in the regulation of endothelial function, its transcriptional regulation remains of great interest, but it remains incompletely defined. Some recent insights include upregulation of Arg2 by S6K and mTOR activation, and its transcriptional downregulation by pharmacological inhibition with rapamycin (Yepuri et al., 2012 Aging Cell, 11:1005-1016). Additionally, epigenetic modification such as methylation of the Arg2 promoter may regulate its transcription (Krause et al., 2013 Epigenetics, 8:944-952).

Interest in epigenetic mechanisms that regulate gene expression is growing. Histone modifications are critical for transcriptional activity, and histone acetylases and deacetylases allow gene expression to be exquisitely regulated through chromatin remodeling. An increase in histone acetylation reduces DNA histone binding, and this allows greater access for DNA transcription factors. Deacetylation has the opposite effects. Although the role of histone deacetylases (HDACs) in tumorigenesis is well established and HDAC inhibitors are being tested as drugs for the treatment of cancer (Johnstone et al., 2002 Nat Rev Drug Discov, 1:287-299), prior to the invention described herein, the role of HDACs in the regulation of endothelial proteins and function was not well established (Dje N′Guessan P et al., 2009 Arterioscler Thromb Vasc Biol, 29:380-386). There are 18 different HDACs that are classified into 4 groups: Class I (HDACs 1,2,3, and 8), Class II (HDACs 4,5,6,7,9, and 10), Class III (SIRT1-7), and Class IV (HDAC11).

As described in detail below, it was examined whether HDACs are critical regulators of endothelial Arg2 expression and whether modulation of HDACs would impact endothelial function. The data presented herein demonstrate that HDAC2 regulates Arg2, HDAC2 downregulation leads to endothelial dysfunction, and overexpression of HDAC2 improves endothelial function in an Arg2-dependent fashion.

The table presented below provides a list of nonstandard abbreviations and acronynms used herein.

TABLE 4
Nonstandard Abbreviations and Acronyms

| | |
|---|---|
| ABH | amino-2-borono-6-hexanoic acid |
| Arg2 | arginase 2 |
| eNOS | endothelial nitric oxide synthase |
| HAECs | human aortic endothelial cells |
| HDAC | histone deacetylase |
| OxLDL | oxidized low-density lipoprotein |
| SNP | sodium nitroprusside |
| TSA | trichostatin A |

The Examples provided below elucidate the role of HDAC2 in the regulation of Arg2 expression. Also described herein is the observation that OxLDL activation of Arg2 in HAECs has a bimodal time line. Increases in arginase activity occurring as early as minutes to hours are attributable to posttranslational modifications of Arg2 and resulting changes in its subcellular compartmentalization (See also, Ryoo et al., 2006 Circ Res, 99:951-960).

As described below, Arg2 mRNA levels rose as early as 4 hours after OxLDL exposure, a delayed peak in Arg2 protein levels occurred beginning at 12 hours, and these changes were ablated by Actinomycin D and cycloheximide, respectively. The data presented herein provide a mechanism for the second and longer term of these 2 effects, whereby OxLDL triggers the release of the Arg2 promoter from the negative epigenetic transcriptional regulation exerted by HDAC2. This leads to an increase in Arg2 transcription and translation and is followed by substantial increments in Arg2 uncoupling of eNOS that begin 12 to 18 hours later, and may have a much longer duration. HDAC1 regulation of Arg1 has been reported in macrophages (albeit in an activating manner), and several groups have reported a mixture of up- and downregulation by HDACs in genes with promoters containing cAMP response elements (Haffner et al., 2008 J Cell Biochem, 103:520-527; and Fass et al., 2003 J Biol Chem, 278:43014-43019). Fass proposed a model for the constraint of NUR77 gene transcription by HDAC in which binding of the promoter to the preinitiation complex is prevented solely by HDAC activity; TSA and other HDAC inhibitors, therefore, trigger NUR77 transcription by inhibiting HDAC activity by either direct or indirect means.

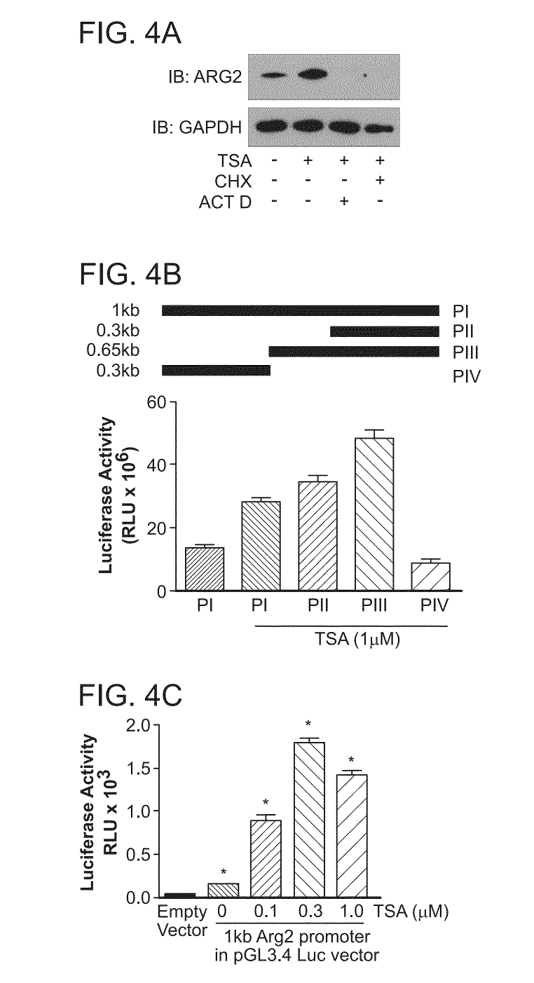

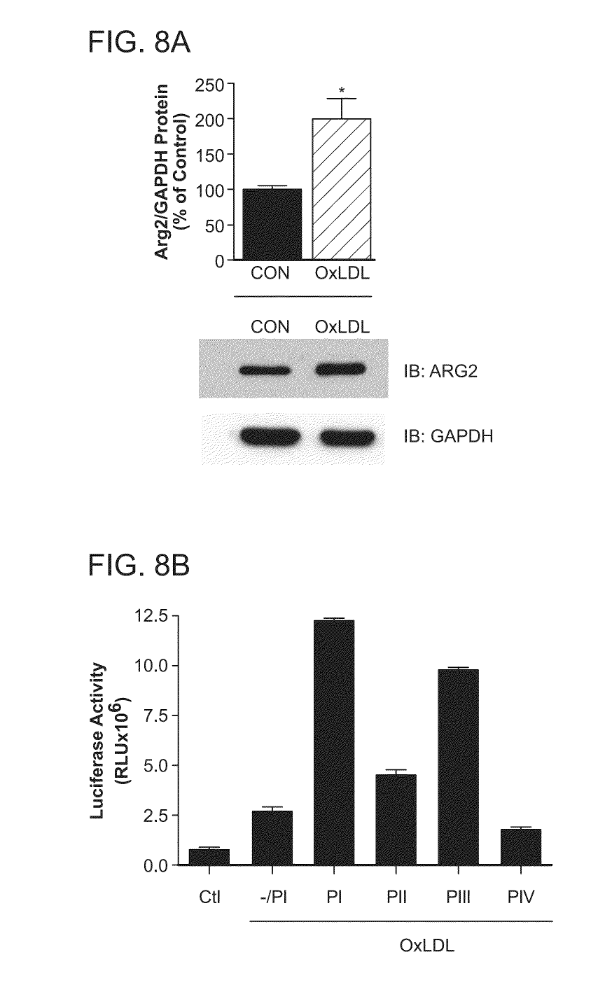

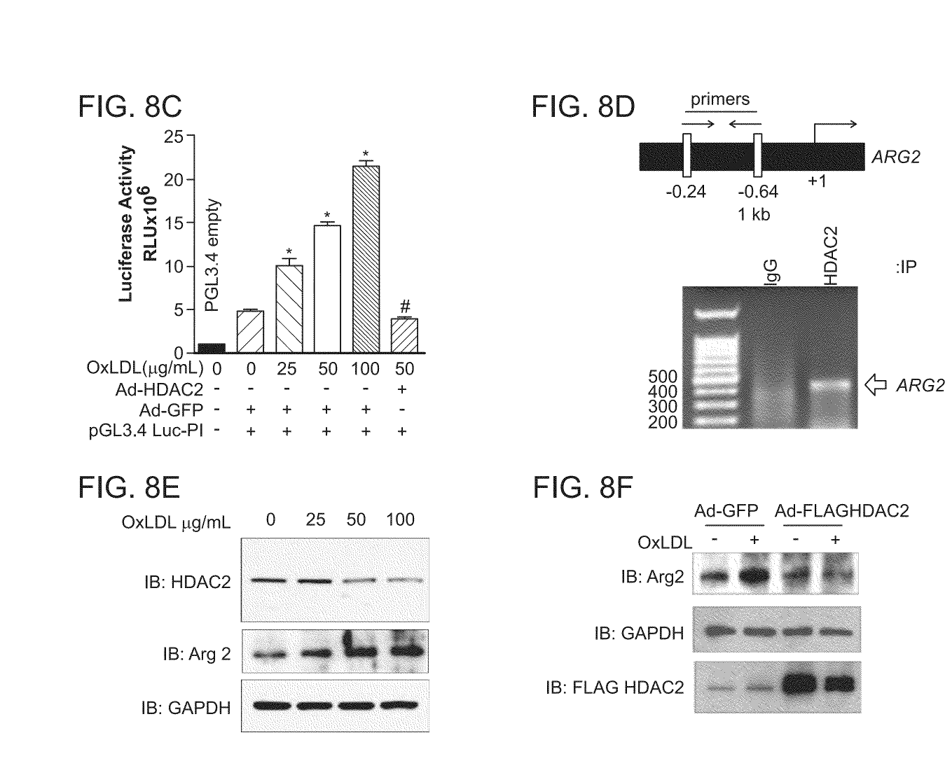

The findings presented herein show that TSA and OxLDL interact with the same regions of the Arg2 promoter (FIG. 4B, FIG. 4C, FIG. 8B, and FIG. 8C) and that the Arg2 promoter eluted with immunoprecipitated HDAC2 in a ChIP assay (FIG. 8D) suggest that the transcriptional activation of Arg2 by OxLDL occurs via HDAC2 inhibition. Indeed, it has been determined that atherogenic stimuli such as OxLDL and flow modulate HDAC2 expression (Dje N′Guessan P et al., 2009 Arterioscler Thromb Vasc Biol, 29:380-386; and Lee et al., 2012 Proc Natl Acad Sci USA, 109:1967-1972). These findings are consistent with the results presented herein in which dose-dependent decreases in HDAC2 expression and simultaneous increases in Arg2 expression were observed with increasing concentrations of OxLDL (FIG. 8E). The findins presented herein indicate that OxLDL also inhibits HDAC2 activity and thereby removes the block that HDAC2 activity presents for Arg2 promoter binding to its preinitiation complex. This scenario gains credence from recent precision mapping of RNA polymerase, which indicates that promoter initiation complexes can extend up to 30 base pairs from the transcription start site and physically contact pausing complexes until these tethers are released (Kwak et al., 2013 Science, 339:950-953). Indeed, as shown in FIG. 8C, a dose-dependent relationship was identified between OxLDL concentrations and activation of the Arg2 promoter. Furthermore, OxLDL has been implicated as a potent inhibitor of Class III HDAC SIRT1 in vascular smooth muscle cells found in atherosclerotic lesions and has been shown to regulate both SIRT3 and aldehyde dehydrogenase 2 activity in HAECs (Gorenne et al., 2013 Circulation, 127:386-396; and Wei et al., 2013 Biochim Biophys Acta, 1833:479-486). A similar pattern of oxidative influence over epigenetic transcriptional regulation is seen in redox modulation of the HDAC control over lineage commitment events in neural progenitor cells (Prozorovski et al., 2008 Nat Cell Biol, 10:385-394).

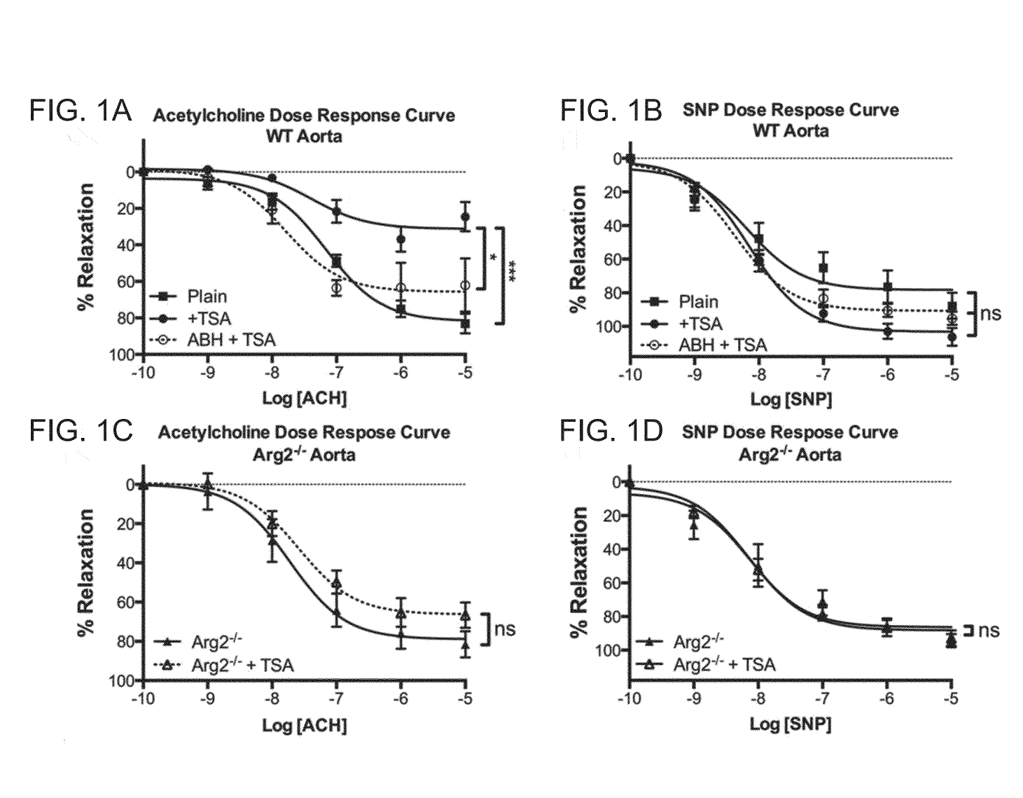

The notable finding and a major impetus for the experiments presented herein is that the attenuated endothelial-dependent vascular relaxation, caused by the broad-spectrum HDAC inhibitor TSA, is almost completely rescued by pharmacological inhibition or genetic knockout of Arg (FIG. 1). Aortic rings preincubated with Class I specific HDAC inhibitor, MGCD0103 (which is highly specific for HDAC1 and HDAC2), also exhibited significantly attenuated vascular relaxation, a pathophysiologic consequence that is highly consistent with the molecular findings described herein.

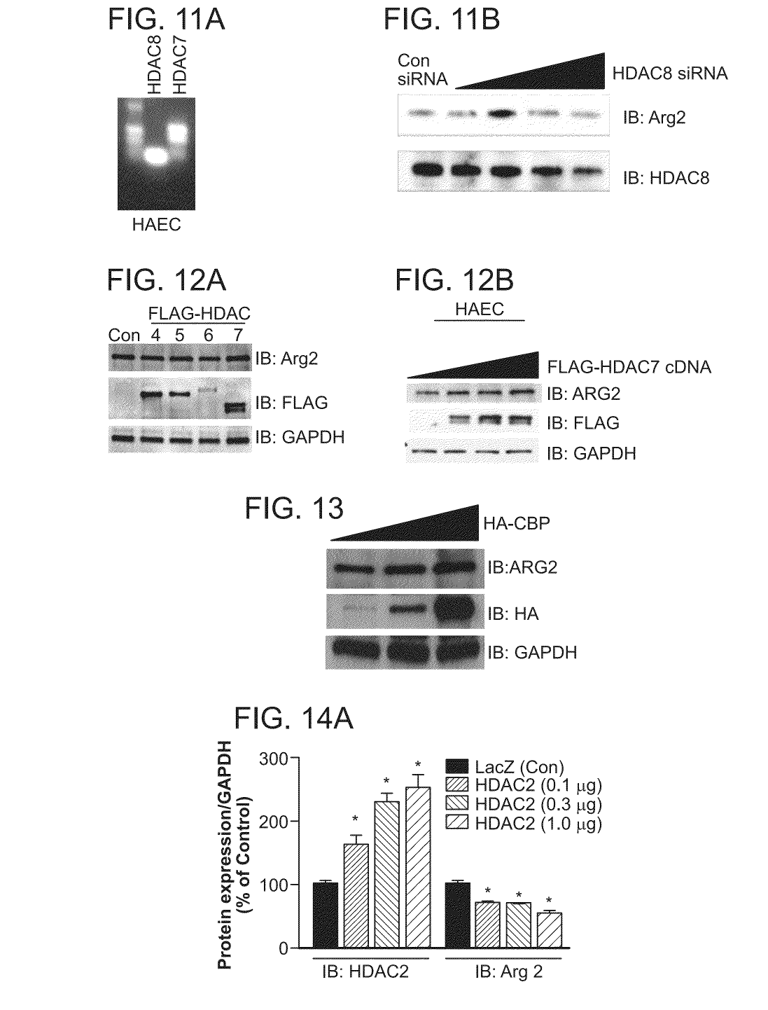

The demonstration of HDAC8 expression in vascular endothelial cells (FIG. 11A) is the first report of its kind. HDAC8 has been implicated in many biological contexts including tumorigenesis, myocardial hypertrophy, and gene silencing during craniofacial development (Helin et al., 2013 Nature, 502:480-488; Ito et al., 2006 J Exp Med, 203:7-13; Barnes P J, 2013 Nat Rev Drug Discov, 12:543-559; and Cosio et al., 2004 J Exp Med, 200:689-695). Prior to the results presented herein, its role in the regulation of endothelial transcription had not been determined.

Targeted drug development for atherogenesis has included both preventive and remedial strategies and has largely focused on decreasing plaque burden. The data presented herein, along with wider experience in preclinical studies in cancer biology indicate that transcriptional regulators such as HDAC2 offer a safer and more effective way forward (Helin et al., 2013 Nature, 502:480-488). For example, described herein is a small molecule strategy that maintains the inhibition of Arg2 transcription by upregulating/activating HDAC2 activity in the specific context of the Arg2 promoter. Indeed, in the context of chronic obstructive pulmonary disease (COPD), resistance to the anti-inflammatory response to corticosteroids may be explained by an oxidative and nitrosylation stress-induced reduction in HDAC2 expression (Ito et al., 2006 J Exp Med, 203:7-13; and Barnes P J, 2013 Nat Rev Drug Discov, 12:543-559). Low-dose theophylline (1,3-dimethylxanthine) increases HDAC2 activity and expression in alveolar macrophages and restores teroid responsiveness (Cosio et al., 2004 J Exp Med, 200:689-695). HDAC2 enhancers that are already widely available, such as methylxanthines, are utilized in the methods described herein.

Despite the emphasis on lifestyle risk management and the advent of statins, atherosclerotic cardiovascular disease remains the leading cause of morbidity and mortality in the West. HDACs that control the expression of various genes by removing an acetyl group from histones are emerging as potential targets in cancer therapeutics and cardiovascular disease. As described herein, inhibition of HDAC2 leads to upregulation of Arg2 and reciprocal impairment of endothelial nitric oxide production and endothelial function. The demonstration that HDAC inhibition leads to profound endothelial dysfunction offers a potential note of caution regarding the use of nonspecific HDACs as cancer therapeutics. Furthermore, the identification of a single HDAC, HDAC2, as a transcriptional regulator of endothelial Arg2 and vascular homeostasis, suggests that this enzyme is a critical node in the modulation of endothelial health.

### Pharmaceutical Therapeutics

The invention provides pharmaceutical compositions for use as a therapeutic. In one aspect, the composition is administered systemically, for example, formulated in a pharmaceutically-acceptable buffer such as physiological saline. Preferable routes of administration include, for example, instillation into the bladder, subcutaneous, intravenous, intraperitoneal, intramuscular, or intradermal injections that provide continuous, sustained levels of the composition in the patient. Treatment of human patients or other animals is carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia or infection. Generally, amounts will be in the range of those used for other agents used in the treatment of other diseases associated with neoplasia or infection, although in certain instances lower amounts will be needed because of the increased specificity of the compound. A compound is administered at a dosage that enhances an immune response of a subject, or that reduces the proliferation, survival, or invasiveness of a neoplastic cell as determined by a method known to one skilled in the art.

### Formulation of Pharmaceutical Compositions

The administration of compositions for the treatment of a condition associated with endothelial dysfunction may be by any suitable means that results in a concentration of the therapeutic that, combined with other components, is effective in ameliorating, reducing, or stabilizing a condition associated with endothelial dysfunction. The composition may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, intravesicularly or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of compound used in mice or nonhuman primates, as a skilled artisan recognizes it is routine in the art to modify the dosage for humans compared to animal models. In certain embodiments it is envisioned that the dosage may vary from between about 0.1 μg compound/kg body weight to about 5000 μg compound/kg body weight; or from about 1 μg/kg body weight to about 4000 μg/kg body weight or from about 10 μg/kg body weight to about 3000 μg/kg body weight. In other embodiments this dose may be about 0.1, 0.3, 0.5, 1, 3, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 μg/kg body weight. In other embodiments, it is envisaged that doses may be in the range of about 0.5 μg compound/kg body weight to about 20 μg compound/kg body weight. In other embodiments the doses may be about 0.5, 1, 3, 6, 10, or 20 mg/kg body weight. Of course, this dosage amount may be adjusted upward or downward, as is routinely done in such treatment protocols, depending on the results of the initial clinical trials and the needs of a particular patient.

Pharmaceutical compositions are formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Kits or Pharmaceutical Systems

Pharmaceutical compositions may be assembled into kits or pharmaceutical systems for use in treating a condition associated with endothelial dysfunction. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the kit.

### Arginase 2

Arg2 is a critical target in atherosclerosis because it controls endothelial nitric oxide, proliferation, fibrosis, and inflammation. Prior to the invention described herein, regulators of Arg2 transcription in the endothelium had not been characterized. The goal of the experiments described herein was to determine the role of specific histone deacetylases (HDACs) in the regulation of endothelial Arg2 transcription and endothelial function.

As described in detail in the examples that follow, the HDAC inhibitor trichostatin A increased levels of Arg2 mRNA, protein, and activity in both human aortic endothelial cells and mouse aortic rings. These changes occurred in both time- and dose-dependent patterns and resulted in Arg2-dependent endothelial dysfunction. Trichostatin A and the atherogenic stimulus oxidized low-density lipoprotein enhanced the activity of common promoter regions of Arg2. HDAC inhibition with trichostatin A also decreased endothelial nitric oxide, and these effects were blunted by arginase inhibition. Nonselective class I HDAC inhibitors enhanced Arg2 expression, whereas the only selective inhibitor that increased Arg2 expression was mocetinostat, a selective inhibitor of HDACs 1 and 2. Additionally, mouse aortic rings preincubated with mocetinostat exhibited dysfunctional relaxation. As described in the Examples below, overexpression of HDAC2 (but not HDAC 1, 3, or 8) cDNA in human aortic endothelial cells suppressed Arg2 expression in a concentration-dependent manner, and siRNA knockdown of HDAC2 enhanced Arg2 expression. Chromatin immunoprecipitation indicated direct binding of HDAC2 to the Arg2 promoter, and HDAC2 overexpression in human aortic endothelial cells blocked oxidized low-density lipoprotein-mediated activation of the Arg2 promoter. Finally, overexpression of HDAC2 blocked oxidized low-density lipoprotein-mediatedvascular dysfunction.

Thus, the results presented herein demonstrate that HDAC2 is a critical regulator of Arg2 expression and thereby endothelial nitric oxide and endothelial function. Overexpression or activation of HDAC2 represents a therapy for endothelial dysfunction in a variety of settings, including artherosclerosis, pulmonary hypertension, vasculitis, and hypertension.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1

### Materials and Methods

The following materials and methods were utilized for the experiments described herein (See also, Pandey et al., 2014 Arterioscler Thromb Vasc Biol, 34:1556-1566, incorporated herein by reference).

### Animals and Reagents

C57BL6 mice were purchased from Jackson Laboratory. Ox-LDL that were prepared by the reaction of LDL and CuSO4 was purchased from Intracel Co (Frederick, Md.). All HDAC inhibitors were purchased from Selleckchem (Houston, Tx) except TSA, which was purchased from Cell Signaling. Unless otherwise stated, all reagents were obtained from Sigma. HDAC2,HDAC8 siRNA was purchased from Qiagen. HDAC2 antibody was purchased from (ABGENT). Arginase 2 antibody was purchased from Sigma (St. Louis, Mo.).

### Cell Culture

HAEC were maintained in ECM culture medium (Science Cell Research Laboratories, Carlsbad, Calif.) according to the supplier's protocol. 293 cells were transfected using Lipofectamine 2000 (Invitrogen) as per manufacturer's protocol and HAEC were transfected using Amaxa transfection system (Lonza).

### RTPCR and Real Time PCR

Total RNA was extracted from segments of mice aortas and HAEC using Trizol and PureLink (Invitrogen). RNA was then reverse transcribed with oligo dT primers to obtain cDNA and quantitative real time PCR (Applied Biosystems) was performed using SYBR Green Supermix mix (Applied Biosystems) whereas semiquantitative RTPCR was performed using conventional Biorad PCR machine and the following primer sets:

| Mouse Arg2: | |
|---|---|
| (SEQ ID NO: 1) | |
| Forward: | 5′-GGG CCC TGA AGG CTG TAG-3′, |
| (SEQ ID NO: 2) | |
| Reverse: | 5′-AAT GGA GCC ACT GCC ATC-3′, |
| Human Arg2: | |
| (SEQ ID NO: 3) | |
| Forward: | 5′-GGG CCC TGA AGG CTG TAG-3′, |
| (SEQ ID NO: 4) | |
| Reverse: | 5′-AAT GGA GCC ACT GCC ATC-3′, |
| Human HDAC8: | |
| (SEQ ID NO: 5) | |
| Forward: | 5′-AAC ACG GCT CGA TGC TGG-3′, |
| (SEQ ID NO: 6) | |
| Reverse: | 5′-CCA GCT GCC ACT TGA TGC-3′ |
| Human HDAC7: | |
| (SEQ ID NO: 7) | |
| Forward: | 5′-CCC AGC AAA CCT TCT ACC A-3′ |
| (SEQ ID NO: 8) | |
| Reverse: | 5′-AAG CAG CCA GGT ACT CAG G-3′. |

Real Time PCR data are expressed as “fold change”, which is calculated as 2-ΔΔCt. This is derived as follows: Delta Ct (ΔCt) denotes the difference in Ct values between the gene of interest (ARG2 in this case) and the housekeeping gene 18s; Delta Delta Ct (ΔΔCt) denotes the difference in “Delta Ct” values between data from samples that are untreated and those that are treated groups with TSA, and this is the ‘fold change’.

DNA constructs and Adenoviruses: FLAG-tagged HDAC2 was constructed by PCR using untagged HDAC2 purchased from the Arizona State University plasmid repository as a template using and following primers: Forward 5′-CAC CAT GGA TTA CAA GGA TGA CGA CGA TAA GG-3′ (SEQ ID NO: 9); Reverse, 5′-TTA CAA GGG GTT GCT GAG CTG TTC TGA TTT GGT TCC-3′ (SEQ ID NO: 10). The PCR product was cloned into pcDNA3.1 using directional topo cloning (Invitrogen) Adenoviruses encoding FLAG-HDAC2 was constructed by subcloning into entry PENTR1a vector using BamH1 and EcorV restriction enzymes followed by LR recombination with destination vector PDEST. The HDAC2-PDEST DNA was digested with PacI, ethanol precipitated and transfected into 293 HEK cells. After cytopathic effect (CPE), adenoviruses were collected and purified via three freeze-thaw cycles and a Millipore adenovirus purification Kit.

Adenovirus Encoding shRNA

Ad-shNontargeted, Ad-shArg1 and Ad-shArg2 encoded viruses were generated using a pAdBLOCK-iT kit (Life Sciences). Briefly, oligonucleotides targeting 2 different regions of Human Arg1 and Arg2 and non-targeted sequence were designed with proprietary software from Life Sciences and cloned into pU6-ENTR. Sequences used were as follows.

Non targeted:Top, 5′-CAC CGA TGG ATT GCA CGC AGG TTC TCG AAA GAA CCT GCG TGC AAT CCA TC-3′ (SEQ ID NO: 11); Bottom, 5′-AAA AGA TGG ATT GCA CGC AGG TTC TTT CGA GAA CCT GCG TGC AAT CCA TC-3′ (SEQ ID NO: 12). Arglsh#A: Top, 5′-CAC CGG GAT TAT TGG AGC TCC TTT CCG AAG AAA GGA GCT CCA ATA ATC CC-3′ (SEQ ID NO: 13); Bottom, 5′-AAA AGG GAT TAT TGG AGC TCC TTT CTT CGG AAA GGA GCT CCA ATA ATC CC-3′ (SEQ ID NO: 14); Arg1sh#B; top, 5′-CAC CGG AGA CAA AGC TAC CAC ATG TCG AAA CAT GTG GTA GCT TTG TCT CC-3′ (SEQ ID NO: 15); bottom, 5′-AAA AGG AGA CAA AGC TAC CAC ATG TTT CGA CAT GTG GTA GCT TTG TCT CC-3′ (SEQ ID NO: 15); Arg2sh#A: top, 5′-CAC CGG TTC TTT AGC TGT CAC TTA GCG AAC TAA GTG ACA GCT AAA GAA CC-3′ (SEQ ID NO: 16); bottom, 5′-AAA AGG TTC TTT AGC TGT CAC TTA GTT CGC TAA GTG ACA GCT AAA GAA CC (SEQ ID NO: 17); Arg2sh#B: top, 5′-CAC CGC ATT CCA TCC TGA AGA AAT CCG AAG ATT TCT TCA GGA TGG AAT GC-3′ (SEQ ID NO: 18); bottom, 5′-AAA AGC ATT CCA TCC TGA AGA AAT CTT CGG ATT TCT TCA GGA TGG AAT GC-3′ (SEQ ID NO: 19); HDAC2sh #A: top, 5′-CAC CGC AGA TGC AGA GAT TTA ATG TCG AAA CAT TAA ATC TCT GCATCT GC-3′ (SEQ ID NO: 20); bottom, 5′-AAA AGC AGA TGC AGA GAT TTA ATG TTT CGA CAT TAA ATC TCT GCA TCT GC-3′ (SEQ ID NO: 21); HDAC2sh #B: top, 5′-CAC CGG AGA AGG AGG TCG AAG AAA TCG AAA TTT CTT CGA CCT CCT TCT CC-3′ (SEQ ID NO: 22); bottom, 5′-AAA AGG AGA AGG AGG TCG AAG AAA TTT CGA TTT CTT CGA CCT CCT TCT CC-3′ (SEQ ID NO: 23).

The resulting pU6-sh-Nontargeted, pU6-ArglshRNA and pU6-Arg2shRNA plasmids were tested for function in transient transfection experiments with 293A cells. The constructs showing the greatest inhibition were LR recombined with pAD/BLOCK-iTDEST (Invitrogen) to generate pAd-shArg1 and pAd-shArg2. Viruses were amplified and purified/concentrated using a Millipore Kit.

### Force Tension Myography

Mouse aorta was isolated and cleaned in ice-cold Krebs-Ringer-bicarbonate solution containing the following (in mM): 118.3 NaCl, 4.7 KCl, 1.6 CaCl2, 1.2 KH2 PO4, 25 NaHCO3, 1.2 MgSO4, and 11.1 dextrose. The aorta was immersed in a bath filled with constantly oxygenated Krebs buffer at 37° C. Equal size thoracic aortic rings (2 mm) were mounted using a microscope, ensuring no damage to the smooth muscle or endothelium. One end of the aortic rings was connected to a transducer, and the other to a micromanipulator. Aorta was passively stretched to an optimal resting tension using the micromanipulator, after which a dose of 60 mM KCl was administered, and repeated after a wash with Krebs buffer. After these washes, all vessels were allowed to equilibrate for 20-30 min in the presence of indomethacin (3 μM). Phenylephrine (1 μM) was administered to induce vasoconstriction. A dosedependent response (1 nM to 10 μM), with the muscarinic agonist, ACH or nitric oxide donor, SNP, was then performed as necessary. The responses were repeated in the presence of inhibitors. Relaxation responses were calculated as a percentage of tension following pre-constriction. Sigmoidal dose-response curves were fitted to data with the minimum constrained to 0. Two to four rings were isolated from each animal and the number of animals in each group (n) was 6.

### Human Arg2 Promoter Assay Using Luciferase Activity

Chromosomal DNA was prepared from HAECs using Trizol reagent as per manufacturer's protocol (Life sciences). Promoter fragments of Arg2 were amplified by PCR using specific primer sets as follows.

PI Forward 5′-GGG CTC GAG TGA GGT GAA ATA AAT TTC AGG AGT TTA-3′ (SEQ ID NO: 24); Reverse 5′-GCC AAG CTT AGC CGG GAG TGA GCG CCA CCG CCC-3′ (SEQ ID NO: 25), 1 Kb; PII Forward 5′-GGG CTC GAG ATA ACC AGC GCT CCC GTT ATT CAG-3′ (SEQ ID NO: 26); Reverse 5′-GCC AAG CTT AGC CGG GAG TGA GCG CCA CCG CCC-3′ (SEQ ID NO: 27), 0.3 Kb; PIII Forward 5′-GGG CTC GAG GTT CAG GAA CCT GGC ATG GGC CGG-3′ (SEQ ID NO: 28); Reverse 5′-GCC AAG CTT AGC CGG GAG TGA GCG CCA CCG CCC-3′ (SEQ ID NO: 29), 0.65 Kb; PIV Forward 5′-GGG CTC GAG TGA GGT GAA ATA AAT TTC AGG AGT TTA-3′ (SEQ ID NO: 30); Reverse 5′-GCC AAG CTT CCG GCC CAT GCC AGG TTC CTG AAC-3′ (SEQ ID NO: 31), 0.3 kb.

These were cloned into the restriction sites for XhoI and HindIII on the pGL3-enhancer vector (Promega Co.). HAECs were transfected with the plasmids using Fugene 6 reagent (Roche Co.) and luciferase activity was measured by Dual-luciferase reporter assay system (Promega Co.). The luciferase activity was reported as relative luciferase units by dividing firefly luciferase activity by Renilla luciferase activity.

Transcription factor (TF) binding consensus sequences in Arg2 promoter sequence were analyzed by using Ensembl, TRANSFAC database (generegulation.com/pub/databases.html#transfac) and MacVector software (Accelrys, San Diego, Calif.).

### Immunoprecipitation and Western Blotting

After 48 hrs of HAEC cells transfection, cells were lysed in ice cold RIPA lysis buffer consisting of 20 mM Tris-HCl at pH 7.5, 150 mM NaCl, 1 mM EDTA, 1 mM EGTA, 1% NP40, 1% sodium deoxycholate, 1 mM Na3VO4, 2.5 mM sodium pyrophosphate, 1 mM 3-glycerophosphate, 1 μg/mL leupeptin, and a 1:1000 diluted protease inhibitor cocktail (Sigma). For immunoprecipitation studies, whole cell lysate lysates were centrifuged at 14,000×g and supernatants were precleared by incubation with Protein A/G-agarose beads for 2 h at 4° C. with rocking. Agarose beads were then pelleted by centrifugation at 1,000×g. FLAG-Arg2 in precleared lysates were immunoprecipitated by incubation overnight at 4° C. with rocking following addition of anti-FLAG antibody (10 μl). Immune complexes were eluted in 2×SDS sample buffer by boiling for 5 minutes before loading into SDS-PAGE. Western blotting analysis was performed by transferring the SDS gel onto a PVDF membrane and visualized using secondary antibodies conjugated to alkaline phosphatases.

### Arginase Activity Assay

HAEC were lysed using RIPA buffer (50 mM of Tris.HCl, pH 7.5, 0.1 mM EDTA, 0.1% Triton X-100, and protease inhibitor), and centrifuged for 30 min at 14,000 g at 4° C. 50 μL (50-100 μg) of supernatant was added to 75 μl of 10 mM MnCl2 made in Tris-HCl (50 mM, pH 7.5) and heat activated by incubating at 55-60° C. for 10 min. 50 μL of L-Arginine that was prepared in 300 mM Tris-HCL, pH 9.7 were then added to the activated cell lysates to achieve final concentrations of 150 mM and incubated at 37° C. for 3 hrs with shaking. The reaction was stopped by adding 400 μL of an acid solution (H2SO4: H3PO4: H20=1:3:7). For colorimetric determination of urea, α-isonitrosopropiophenone (25 μL, 9% in ethanol) was added and further heated at 100° C. for 45 min. Samples were allowed to cool at room temperature in the dark for 10 min and urea concentration was determined by measuring absorbance at 550 nm using a spectrophotometer.

### Chromatin Immunoprecipitation (ChIP)

ChIP was performed using Magna ChIP kit from Millipore as per manufacturer's protocol. Briefly, Confluent HAEC were incubated with 1% formaldehyde at 37° C. for 10 minutes to crosslink Histones and DNA. The crosslinked complexes were subjected to shearing with sonication and immunoprecipitated with anti-HDAC2 antibody. After reverse crosslinking, purified and immunoprecipitated DNA was amplified using Arg2 promoter specific primers as shown in methods for Arg2 promoter assay.

### Measurements of Nitric Oxide Production

Nitric Oxide levels in HAEC were determined using DAF-DA as per manufacture instructions (Life Sciences). Briefly, cells were loaded with DAF-DA (5 μM) for 20 min and washed 3 times with PBS and incubated for another 30 min in phenol-free media. Fluorescence was determined using a Nikon TE-200 epifluorescence microscope. Images were captured with a Rolera EMC2 camera (Q-Imaging, BC, Canada) with Velocity software (PerkinElmer, Lexington, Mass.). NO production in the cell culture media was also determined by measuring Nitrite levels using a Siever's NO analyzer. NO radicals were measured in cell media collected after 24 hours of treatment using the Oxiselect assay (Cell Biolabs, San Diego, Calif.).

### Statistics

All statistical analyses were performed using Prism 5 for Mac by GraphPad Software Inc. and Microsoft Excel version 14.1.3 statistical analysis software. The results were expressed as mean and standard error (mean±SEM). One-way analysis of ANOVA and the Bonferroni post-hoc test for multiple-comparison were used to compare all experimental data sets groups and pairs of data sets, respectively. A value of p<0.05 was considered statistically significant.

### Example 2

### HDAC Inhibition Impairs Endothelial-Dependent Vascular Relaxation

Previous findings from the study by Rossig have shown that trichostatin A (TSA), a broad-spectrum HDAC inhibitor with promising effects on a variety of human cancer cells, might cause impairment of endothelium-mediated vascular relaxation (Rossig et al., 2002 Circ Res., 91:837-844). This response was attributed to a small decrease in eNOS expression, but the amount of this decrement raises the question of whether other genes that are regulated by HDACs may have contributed to the attenuated vascular relaxation responses to acetylcholine that Rossig's group observed.

Because Arg2 is a competitive inhibitor of eNOS that impairs endothelial function, Arg2 knockout mice and biochemical inhibitors were used to examine the possibility that Arg2 might mediate the diminished vascular relaxation caused by TSA. Preincubation of aortic rings of wild-type mice with 200 nmol/L of TSA for 18 hours led to impairment of acetylcholine-mediated (endothelium-dependent) vasorelaxation compared with rings treated with vehicle alone (Eₘₐₓ 31.24±7.8 versus 81.9±6.2% for controls; P<0.001, n=6), but this TSA-mediated impairment of vasorelaxation was abolished in wild-type aortic rings that were pretreated with the arginase inhibitor amino-2-borono-6-hexanoic acid (ABH) (Eₘₐₓ 65.7±12.1%; P<0.05; n=6). Preincubation of aortic rings from Arg2−/− mice with TSA (200 nmol/L) showed no impairment of endothelial cell-dependent vasorelaxation compared with vehicle-treated vessels (Eₘₐₓ 66.3±7.54 versus 79.0±10.7% in controls; ns; n=6) (FIG. 1A-FIG. 1D). There was no significant difference in sodium nitroprusside (SNP)-mediated vasorelaxation (an endothelium-independent response because SNP is a NO donor) in aortas that were treated with TSA or MGCD0103 versus the vehicle-only control. These findings indicate that TSA-mediated impairment of vascular relaxation occurs through the upregulation of Arg2 in the intimal endothelium.

### Example 3

### HDACs Regulate Arg2 Expression and Activity

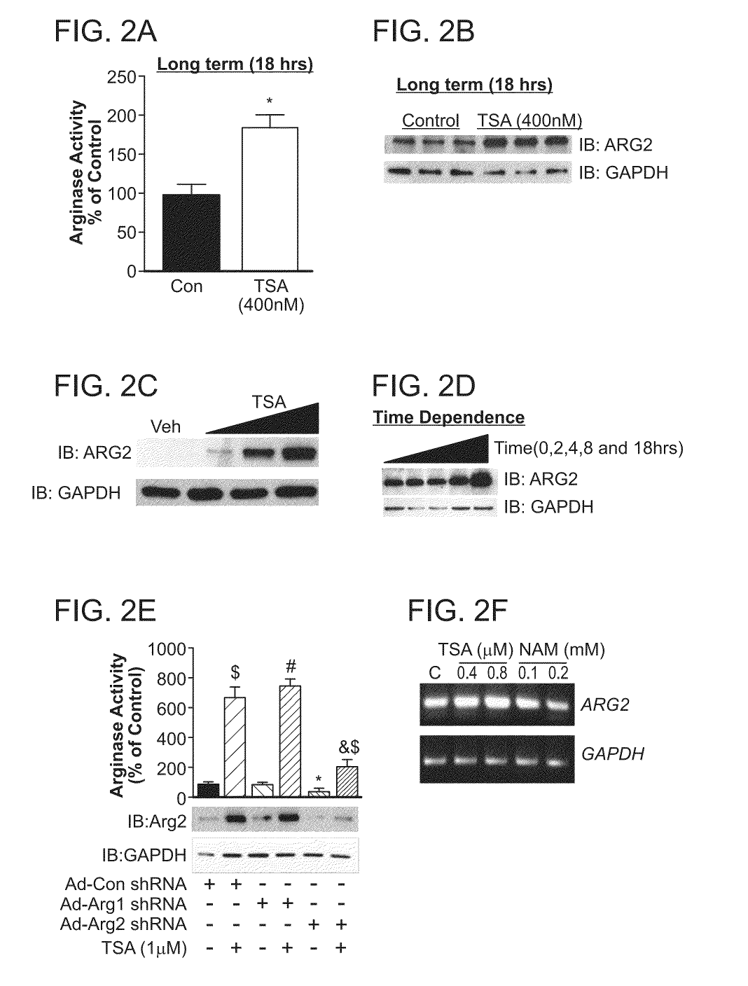

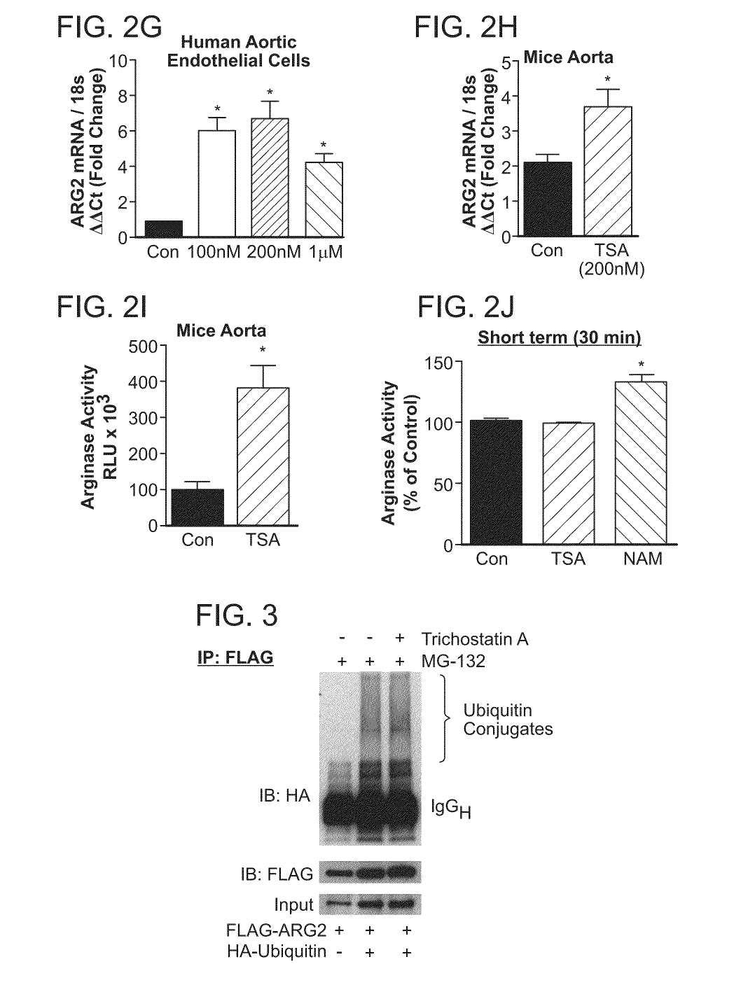

To define and quantify the nature of TSA modulation of Arg2 expression, human aortic endothelial cells (HAECs) were treated with 400 nmol/L TSA for 18 hours as suggested by the manufacturer (Cell Signaling), and arginase activity and protein expression were then measured. TSA increased both total arginase activity and Arg2 expression (FIG. 2A and FIG. 2B). The effect of TSA on Arg2 protein expression was found to be dose dependent. HAECs were incubated with increasing concentrations of TSA (0, 0.1, 0.3, and 1 μmol/L). TSA treatment for 18 hours increased Arg2 in a dose-dependent fashion, with a maximum effect at 1 μmol/L (FIG. 2C and FIG. 10A). The effect was also time dependent with an increase in Arg2 protein expression observed as soon as 8 hours after TSA (1 μmol/L) exposure (FIG. 2D and FIG. 10B). TSA dose and incubation time were 1 μmol/L and 18 hours, respectively, for subsequent experiments with HAECs. Next, adenoviral constructs of Arg1 and Arg2 shRNA were used to confirm that the increase in arginase activity is because of Arg2 rather than Arg1 upregulation. As shown in FIG. 2E, Arg2 shRNA inhibited the TSA-mediated (1 μmol/L) increase in arginase activity, whereas Arg1 shRNA had no effect. To determine whether the TSA effects on increased Arg2 activity and protein expression resulted from changes at the transcriptional level, Arg2 mRNA levels were examined in HAECs and isolated mice aorta that were exposed to TSA using Arg2-specific primers (See, Example 1) for both RT-polymerase chain reaction (FIG. 2F) and real-time polymerase chain reaction reactions. Arg2 mRNA was substantially upregulated in both cultured HAECs and mice aorta that had been preincubated with TSA (FIG. 2F-FIG. 2H). Incubation of isolated segments of mice aortas in TSA (200 nmol/L) also induced a robust increase in arginase activity (FIG. 2I). This reduced concentration of TSA was used for experiments with aorta to protect their fragile intima from toxicity.

The NAD-dependent Sirtuin family (SIRT or Class III) of HDACs has been implicated in endothelial function (Mattagajasingh et al., 2007 Proc Natl Acad Sci USA, 104:14855-14860). To determine whether SIRTs are involved in the regulation of Arg2 expression, HAECs were incubated in the presence of the SIRT inhibitor nicotinamide. No changes were observed in the Arg2 mRNA expression in HAECs after incubation with 0.1 or 0.2 mmol/L of nicotinamide for 18 hours (FIG. 2F). Short-term inhibition (30 minutes) of SIRT with nicotinamide (5 mmol/L) had an activating effect on HAEC arginase activity, whereas TSA (1 μmol/L) had no effect in this time frame as shown in FIG. 2J. This indicates that SIRTs might be important in the short-term regulation of Arg2 activity, and the time course of this effect implies a mechanism involving posttranslational modification.

### Example 4

### HDAC Inhibition by TSA Does Not Affect Arg2 Ubiquitination

Posttranslational modification by ubiquitin subjects proteins to proteasomal degradation and, therefore, affects their abundance. Because both acetylation and ubiquitination target lysine residues of proteins, there is a significant cross talk between these 2 pathways (Yang et al., 2008 Mol Cell, 31:449-461). To determine whether Arg2 is ubiquitinated and whether increased acetylation resulting from exposure to HDAC inhibitors may compete with ubiquitination sites on Arg2 and, thus, increase Arg2 abundance by that mechanism, FLAG was immunoprecipitated from HAECs coexpressing FLAG epitope-tagged Arg2 and HA-tagged ubiquitin and immunoblotted for FLAG. As shown in FIG. 3, bands migrating at higher molecular weights were observed in cells expressing both Arg2 and ubiquitin. This finding is consistent with polyubiquitination of Arg2. However, preincubation with TSA had no effects on the ubiquitination of Arg2, and this excludes ubiquitination as a potential mechanism for TSA-mediated augmentation of Arg2 abundance. This is the first report to show that Arg2 is modified by ubiquitin.

### Example 5

### HDAC Inhibition-Mediated Increases in Arg2 Expression Occur at the Transcriptional Level via Upregulation of Arg2 Promoter Activity

To determine whether TSA affects transcription of Arg2, HAECs were pretreated with actinomycin D and cycloheximide for 1 hour before treatment with TSA (1 μmol/L). TSA failed to increase Arg2 expression in cells treated with either agent (FIG. 4A). Next, it was determined whether inhibition of HDACs affects ARG2 promoter activity. HAECs were transiently transfected with different fragments of a −1 kb region of the human ARG2 promoter (PI) that were cloned into the pGL3 luciferase (LUC) reporter plasmid. The following day, cells were treated with TSA (1 μmol/L) for 8 hours, and luciferase activity was measured using a luciferase kit (Promega). As shown in FIG. 4B, Arg2 promoter activity was increased in cells treated with TSA compared with control cells that were treated with vehicle alone. The −0.3-kb (PII) and −0.65-kb (PIII) fragments of the Arg2 promoter exhibited the highest activity after TSA exposure, whereas the fragment between −1 kb and −0.65 kb, PIV, was not responsive to TSA stimulation. The effect of TSA on the Arg2 promoter was also dose dependent as shown in FIG. 4C. These findings strongly indicate that TSA activates the Arg2 promoter.

### Example 6

### Inhibition of HDACs by TSA Attenuates NO Production

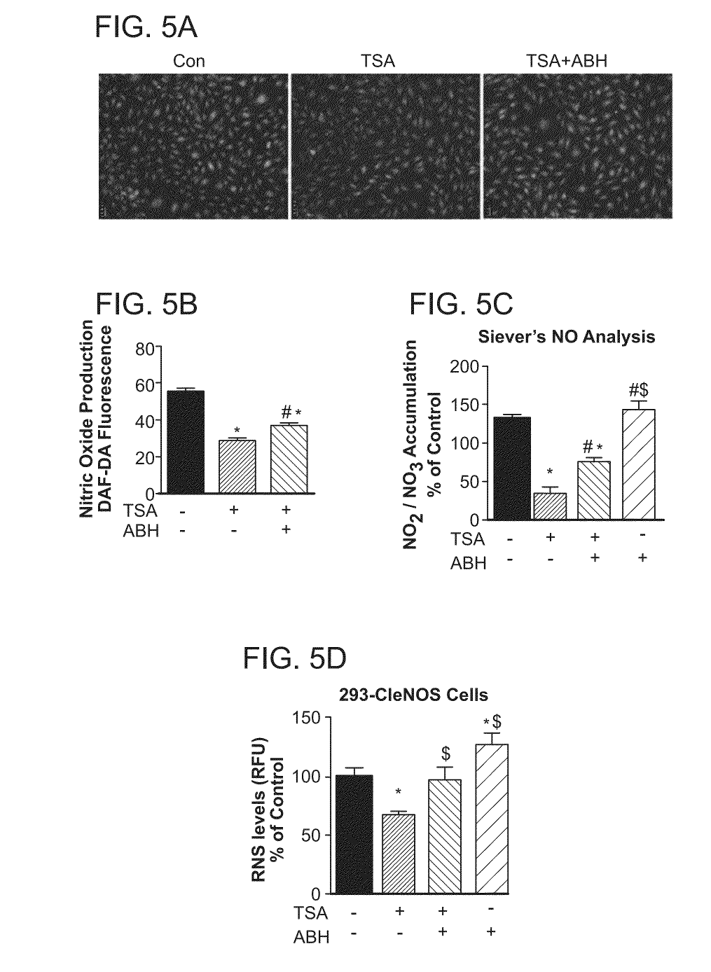

Next, the functional consequences of HDAC inhibition on downstream endothelial cell signaling were evaluated. Because eNOS-derived NO plays a central role in endothelial function, eNOS activity was assessed by measuring NO production using 3 different assays. DAF-DA fluorescence (FIGS. 5A and 5B) and the Siever's NO analyzer (FIG. 5C) were used to determine basal NO production from HAECs challenged with TSA (1 μmol/L) alone and coincubated with ABH (1 μmol/L). As shown in FIGS. 5A and 5B, inhibition of HDAC with TSA significantly decreased NO production in HAECs, and this failed to occur when cells were coincubated with TSA and ABH. A DCFH-DiOxyQ-based fluorescence assay (FIG. 5C) was used to determine NO radical levels in growth medium from human embryonic kidney (HEK) 293 cells that were expressing calcium-insensitive eNOS that were treated with either TSA alone or together with ABH. HEK 293 cells lack native eNOS expression, but do produce abundant Arg2. Cell medium from 293 cells transfected with a control plasmid (LacZ) was used as a control background signal that was subtracted from all the groups containing calcium-insensitive endothelial nitric oxide synthase. TSA decreased NO production from 293 cells expressing calcium-insensitive endothelial nitric oxide synthase. In the presence of ABH, TSA failed to attenuate NO production from 293-calcium-insensitive endothelial nitric oxide synthase cells (FIG. 5C). These findings indicate that the increase in Arg2 that is caused by HDAC inhibition is the major underlying mechanism that drives the diminished eNOS activity with TSA.

### Example 7

### Arg2 Expression is Specifically Regulated by HDAC2

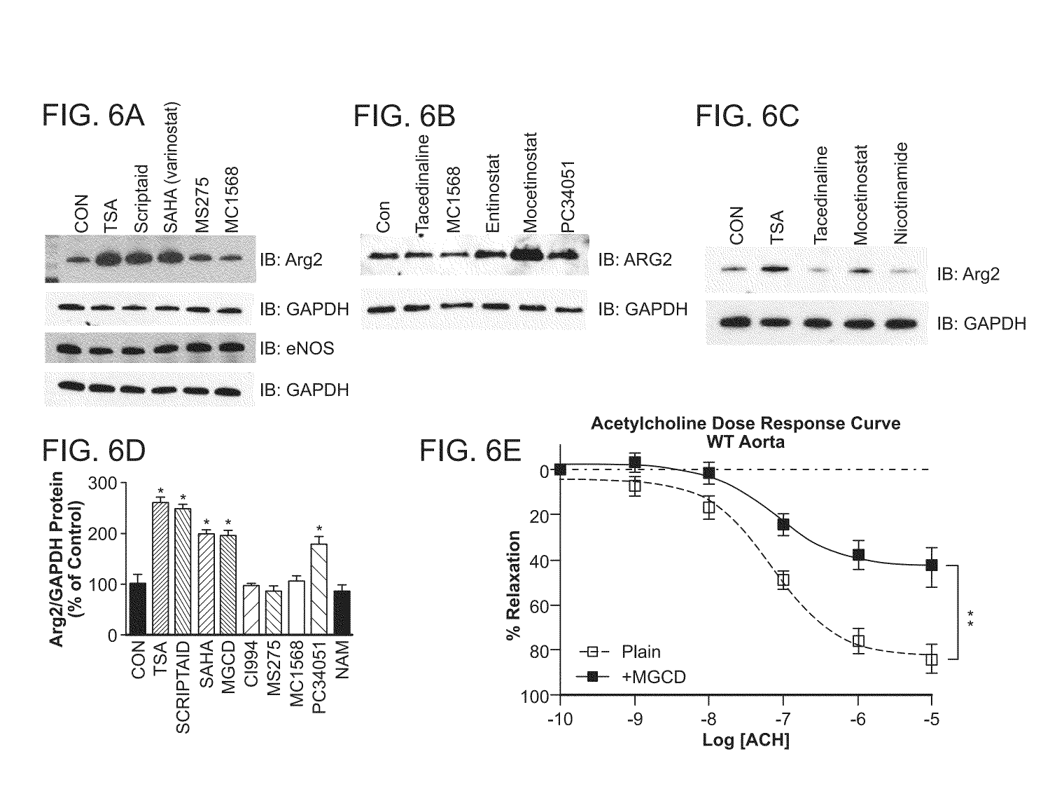

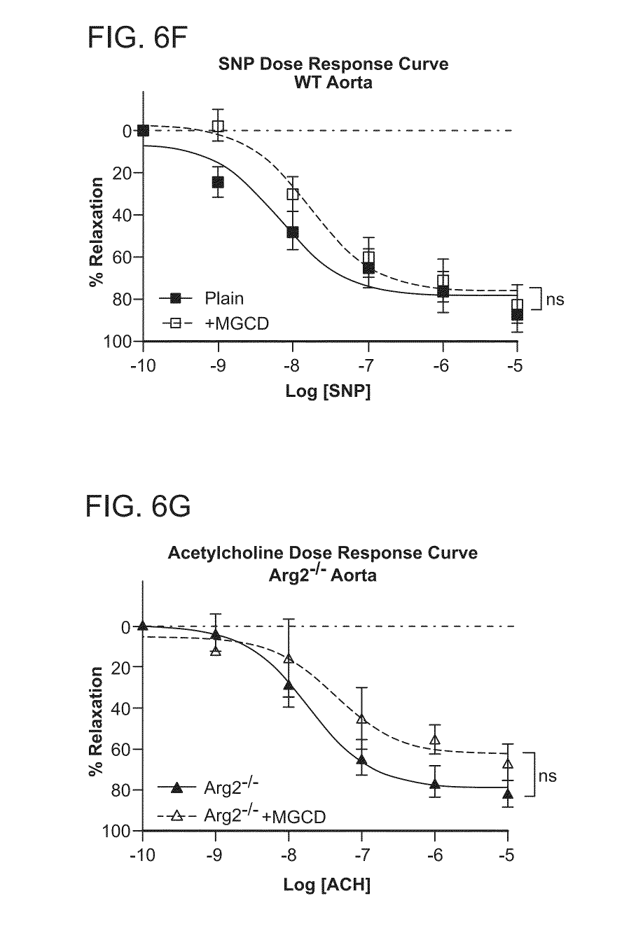

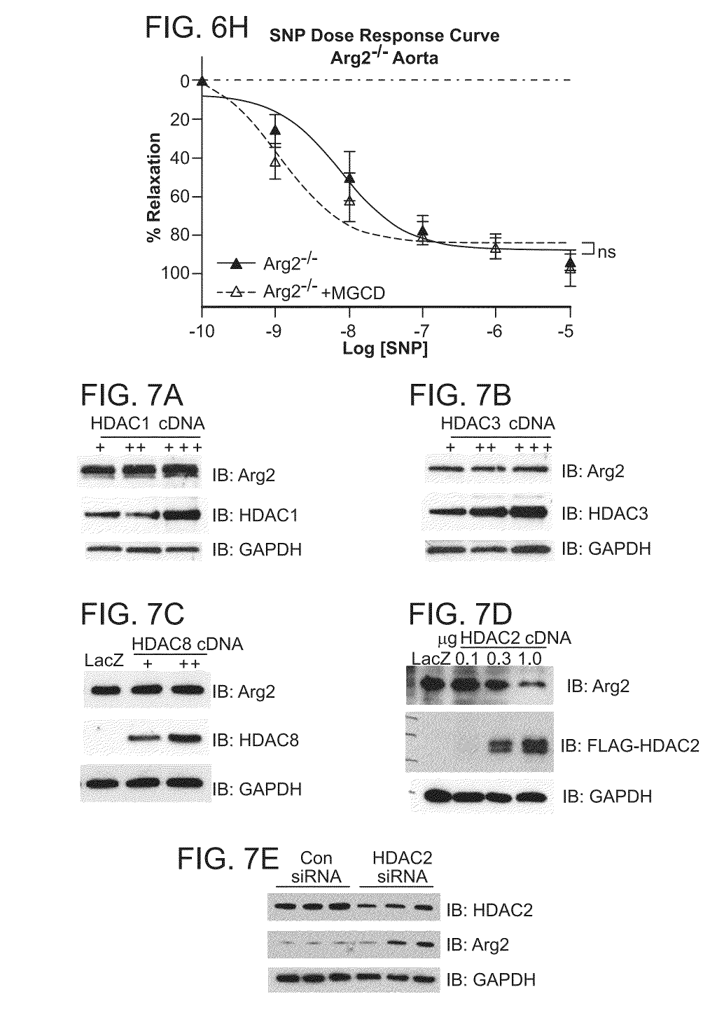

To test whether augmented Arg2 expression was an effect limited to TSA, 2 other broad-spectrum HDAC inhibitors were tested together with TSA: SAHA and Scriptaid. It was found that incubation of HAECs for 18 hours with 1 μmol/L of all 3 agents upregulated Arg2 expression to similar levels (FIG. 6A). In contrast, these inhibitors had negligible effect on eNOS expression (FIG. 6A). To determine the specific HDAC that regulates Arg2 expression, classspecific commercially available pharmacological inhibitors were used (See, Table I for IC50 data).

TABLE 1
IC50 for HDAC Inhibitors Used in Results Presented Herein

| HDAC Inhibitors | IC50 |
|---|---|
| TSA | 1.8 nM (do not block HDAC8 and SIRT) |
| Scriptaid | HDAC1: IC50 = 0.6 μM; HDAC3: IC50 = |
| | 0.6 μM; HDAC8: IC50 = 1 μM |
| SAHA (Suberoylanilide | 10 nM; block Class I and Class II HDACs |
| Hydroxamic Acid) | |
| MGCD0103 | HDAC1 with IC50 of 0.15 μM, 2 to 10 fold |
| (Mocetinostat) | selectivity against HDAC2, 3, and 11, and no |
| | activity to HDAC4, 5, 6, 7, and 8. |
| CI994 (Tacedinaline) | Inhibit HDAC1 with IC50 of 0.57 μM |
| MS275 (Entinostat) | Selective to HDAC1 and HDAC3 with IC50 |
| | of 0.51 μM and 1.7 μM |
| MC1658 | Selective to HDAC1A with IC50 of 100 nM |
| PCI-34051 | Specific HDAC8 inhibitor with IC50 of 10 nM |
| Nicotinamide | IC50 <50 μM |

Nicotinamide (5 mmol/L), a potent Sirtuin (Class III NADdependent HDACs) inhibitor, had no effect on Arg2 mRNA levels (FIG. 2F) or Arg2 protein levels (FIGS. 6C and 6D), suggesting that this family of HDACs is not likely to regulate Arg2 expression. A Class I-specific inhibitor that is selective for HDAC1 and HDAC3, entinostat (MS275, 2 μmol/L), a Class I-specific and HDAC1-selective inhibitor, tacedinaline (CI994, 1 μmol/L), a Class II-specific MC1568 (0.5 μmol/L), and the HDAC6/HDAC8 selective inhibitor-PC34051 (100 nmol/L) all had no substantial effects on endothelial Arg2 expression. In contrast, the Class I specific inhibitor mocetinostat (MGCD0103, 1 μmol/L), which selectively inhibits both HDAC1 and HDAC2, did upregulate Arg2 expression (FIG. 6B-6D). This screening suggested that HDAC2 was the most likely candidate for a direct role in Arg2 regulation. To investigate whether MGCD0103 had similar effects on vascular relaxation as those seen with TSA, isolated mice aortic rings were incubated in MGCD0103 (1 μmol/L) for 18 hours. Vascular reactivity was then measured in the presence of either acetylcholine or SNP. MGCD0103 significantly attenuated endothelial-dependent vascular relaxation compared with vessels treated with vehicle alone (Eₘₐₓ 42.7±8.5 versus 82.8±7.4%; P<0.001; n=6) as shown in FIG. 6D. Endothelium-independent relaxation as assessed by SNP was unaffected by MGCD0103 (FIG. 6E).

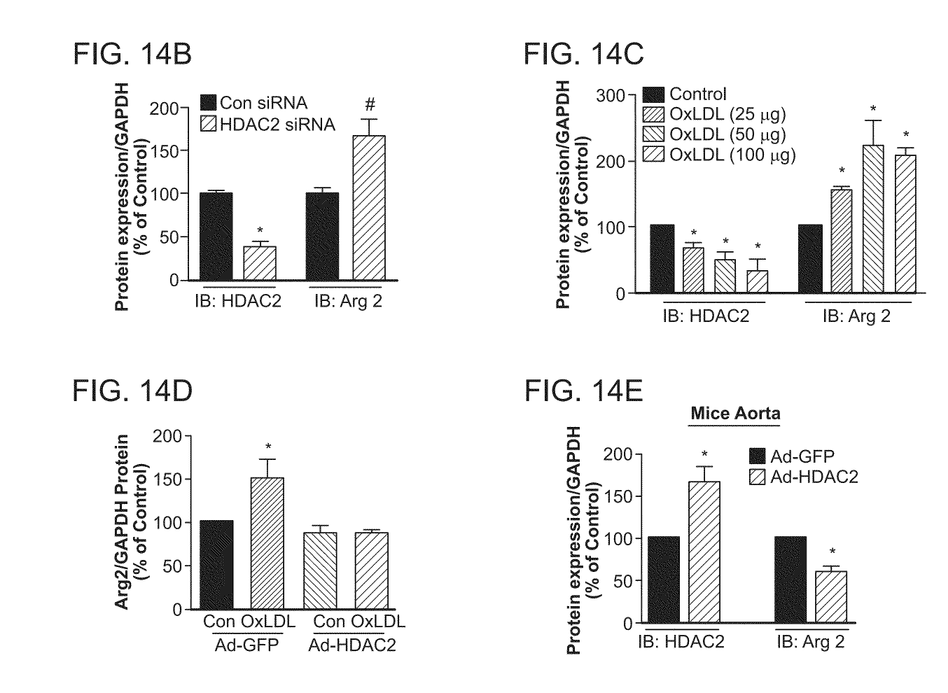

To further determine the role of Arg2 in MGCD0103-mediated vascular dysfunction, dose-dependent vascular relaxation was measured in MGCD0103-treated aortas isolated from Arg2−/− mice treated with either acetylcholine or SNP and found no significant differences in endothelium-dependent relaxation compared with aortas from Arg2−/− that were treated with vehicle alone (Eₘₐₓ 62.4±16.5 versus 79.0±10.7%; ns; n=6) (FIGS. 6F and 6G). Next, HAECs were transfected with increasing concentrations of Class I HDAC (HDAC 1, 2, 3, and 8) cDNAs (0.3 and 1 μg). HDAC 1, 3, and 8 overexpression did not have any effect on Arg2 expression (FIG. 7A-FIG. 7C), whereas HDAC2 overexpression decreased Arg2 expression (FIG. 7D and FIG. 14A). Additionally, no substantial change in Arg2 expression was observed with increased expression of other HDACs that have been implicated in the regulation of endothelial cell function—HDAC 4, 5, 6, and 7 (FIG. 12A and FIG. 12B). To further confirm the involvement of HDAC2 in Arg2 regulation, HDAC2 was silenced in HAECs using siRNA (Qiagen). Arg2 expression was significantly increased with HDAC2 siRNA knockdown (FIG. 7E and FIG. 14B). Because a slight increase in Arg2 expression was observed with PC34051, a specific HDAC8 inhibitor, it was evaluated whether HDAC8 has an impact on Arg2 expression. It was demonstrated that HDAC8 is expressed in HAECs by using RT-polymerase chain reaction (FIG. 11A). HDAC7, which is well known to be expressed by endothelial cells, was used as a positive control. Using siRNA-mediated knockdown and ectopic transfection-mediated overexpression of HDAC8 in HAECs, it was shown that HDAC8 is not an important regulator of Arg2 expression (FIG. 11A, FIG. 11B, and FIG. 7C).

### Example 8

### HDAC2 Effects on Activation of the Arg2 Promoter by OxLDL

It was next examined whether the atherogenic stimulus OxLDL, which is known to increase Arg2 expression (Ryoo et al., 2006 Circ Res., 99:951-960; also demonstrated in FIG. 8A) had any effect on Arg2 promoter activity. Arg2 promoter fragments were cloned into luciferase constructs as illustrated in FIG. 4B. Indeed, OxLDL robustly increased the activity of specific Arg2 promoter fragments (FIG. 8B). Similar to TSA, fragments between −1 kb and 0.3 kb were not responsive to OxLDL (FIG. 8B). This indicated that common Arg2 promoter regions are regulated by atherogenic stimuli and HDACs. To determine whether OxLDL-mediated activation of the Arg2 promoter is a dose-dependent phenomenon, HAECs were transfected with Arg2 (−1 kb) promoter in a luciferase construct. Subsequently, cells were stimulated with increasing concentrations of OxLDL (0, 25, 50, and 100 μg/mL). As shown in FIG. 8C, OxLDL increased Arg2 promoter activity in HAECs in dose-dependent manner. The activating effect of 50 μg/mL of OxLDL on Arg2 promoter activity was completely abolished in HAECs transduced with adenovirus encoding HDAC2. Further, the Arg2 promoter bound to immunoprecipitated HDAC2 from HAEC DNA/protein complexes (FIG. 8D) was amplified using a ChIP assay. To further determine whether OxLDL modulates HDAC2 abundance, HAECs were exposed to increasing doses of OxLDL (0, 25, 50, and 100 μg/mL) and HDAC2 expression was measured. HDAC2 expression was progressively decreased by exposure to 25-100 μg/mL of OxLDL (FIG. 8E and FIG. 14C). These cells simultaneously exhibited a dosedependent increase in Arg2 expression, suggesting a reciprocal regulation of Arg2 by HDAC2 (FIG. 8E, second panel). It was further demonstrated that OxLDL-mediated increases in HAEC Arg2 expression (FIG. 8F) were abolished by HDAC2 overexpression (FIG. 8D and FIG. 14D). This finding is further supported by recent observations that link reduced HDAC2 expression with OxLDL exposure (Dje N′Guessan P et al., 2009 rterioscler Thromb Vasc Biol, 29:380-386). These results strongly indicate that decreased HDAC2 abundance is a critical downstream mechanism for OxLDL-mediated increases in Arg2 expression during atherogenesis. It is possible that other transcriptional regulators may also modulate Arg2 expression under conditions of cellular stress. The Arg2 promoter sequence, candidate regulators, and their potential targets in the Arg2 promoter sequence are summarized in Table II and Table III.

TABLE II
Arginase (−1 kb) promoter sequence
(SEQ ID NO: 32)

TGAGGTGAAATAAATTTCAGGAGTTTATGTAAAAAATTAATTTGGTAGAA
ATAATGTCTAGATTCTTGCATGCCATGGTGTCTTGGTTAAGAACTTGGAA
TGTGGAGTCAGCTCTGCCACTTACAAGCTATGTGGCTCTGGGAAAATTAC
TTATGTCTTTGATCTTTTCTTCGCTTTAAAAAGTAACCTGTGGTATAGAA
TTGGAGGGGTAAAGTTCTTGGAATGCTGAGATTATTAAATTATTATTCGT
TATTAAAACTCTAGTTACTTAGCACTTACAGCTCTTTAGCTTAGAAATAG
TCTAAATACGTTGCTATATTTGCTTTTTTAATAACCAGCGCTCCCGTTAT
TCAGGTAAAGAATTAAAAACATCTAAAAGGTAATTGCTTTGTAAATCCTA
ACCTCTTTCATTCAAAATACCTAAGGTAGATGGCTCTTTATAAACGTGCC
CGGCTCAGAGAGCTCTCATCTATATTATATCTCCGAATCCAAATTTCACT
GATGCCCAGTCTAAGAGTGTTTCCTGGGCTTTGAGCTTTGAGATAGAACG
AAGCAGGTGGATCCTGGCTTTGACTAGGCAGGGCCAGACCAGGCCCGACC
CAGCCAGCCCATCCGTAGAGTCAGCATCAGCCGGGGGTGAAGATTGGTGT
CGGCTCCTGCGTTCCGCCAAGTTCAGGAACCTGGCATGGGCCGGCCGCCT
CCCGCGAAGGGACACACCTGTTGGTCACCGCGCTCTTACTGGCCAGACCC
GGATATTGGCCTTCTGGCGTGAGCGGCAGGGGCATGTCCCTGGCCGGAGA
GCACAGGCCAGAGTGGGGTGACGTCATCGAAGGCACGTCCCAGCCTTGCA
GAGCGGTGGGCGGGGCCTGCGAAGAAGGTGTGCCGGGGGCTGGTTGGAGG
CGCGGGCGGCGGGGCCAATGTGGCGGTGACGTCACGGGGCGGGCGGACGC
TGGCGCGGGTAGGTAAGAGCAGCGGCGGGCGGTGGCGCTCACTCCCGGCT

TABLE III
Transcription factors binding consensus
sequences in Arg2 promoter

| Factor | | |
|---|---|---|
| Name | Sequence | SEQ ID NO: |
| HMGIY | gaaatAAATTtcagg | SEQ ID NO: 33 |
| Fre-ac3 | tttatGTAAAaaatta | SEQ ID NO: 34 |
| ATF2 | TTATGtaa | SEQ ID NO: 35 |
| HNF1 | ttatgtaaaaaATTAAtttgg | SEQ ID NO: 36 |
| SOX10 | cTTTGTa | SEQ ID NO: 37 |
| CDX-2 | TTTATa | SEQ ID NO: 38 |
| HIF-1 alpha | ACGTGc | SEQ ID NO: 39 |
| ZNF333 | ATTAT | SEQ ID NO: 40 |
| p53 | tgaCTAGGcagggccagacc | SEQ ID NO: 41 |
| BEN | CAGCGgca | SEQ ID NO: 42 |
| CPBP | caGGGGC | SEQ ID NO: 43 |
| SREBP | gagtggGGTGAcgtc | SEQ ID NO: 44 |
| CREB1 | ggtgaCGTCAtc | SEQ ID NO: 45 |
| ATF2 | TGACGtca | SEQ ID NO: 46 |
| Sp1 | tgGGCGGggc | SEQ ID NO: 47 |
| Egr-1 | gcGCGGGcgg | SEQ ID NO: 48 |
| RNF96 | ggcgGCGGGg | SEQ ID NO: 49 |
| CPBP | gcGGGGC | SEQ ID NO: 50 |
| CREB1 | ggtgaCGTCAcg | SEQ ID NO: 51 |
| CREB1 | ggTGACGtcacg | SEQ ID NO: 52 |
| ATF2 | TGACGtca | SEQ ID NO: 53 |

Additionally, it was determined that increased expression of a histone acetyl transferase, CBP, which causes global cell acetylation, has no effect on the endothelial Arg2 expression (FIG. 13). This further supports the notion that the transcriptional effect on Arg2 expression involves HDAC2 interaction with the Arg2 promoter, and this effect is not occurring simply by enhanced transcriptional accessibility because of increased global acetylation.

### Example 9

### OxLDL-Mediated Impairment of Vascular Relaxation is Ameliorated by HDAC2 Overexpression

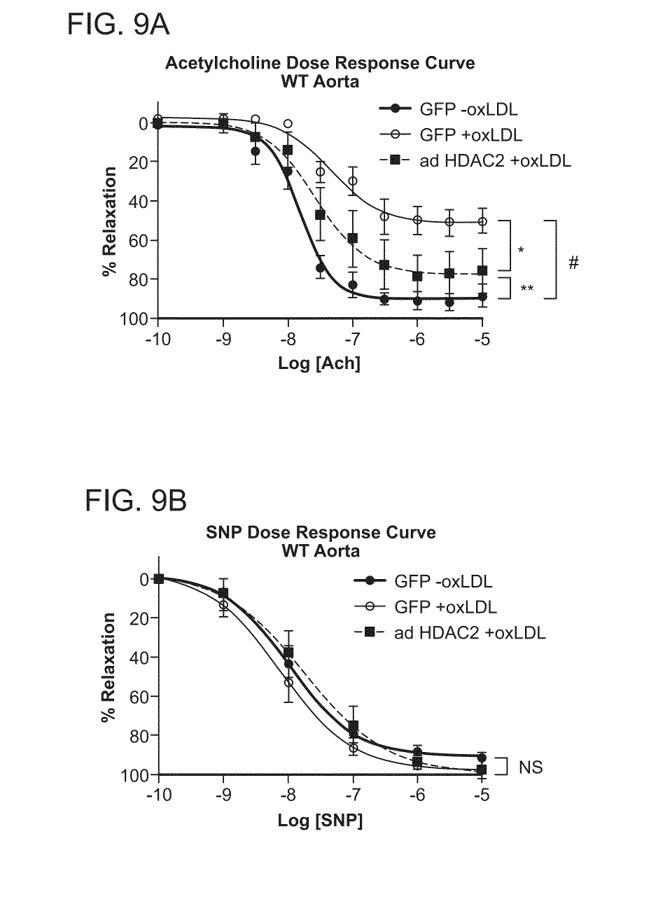

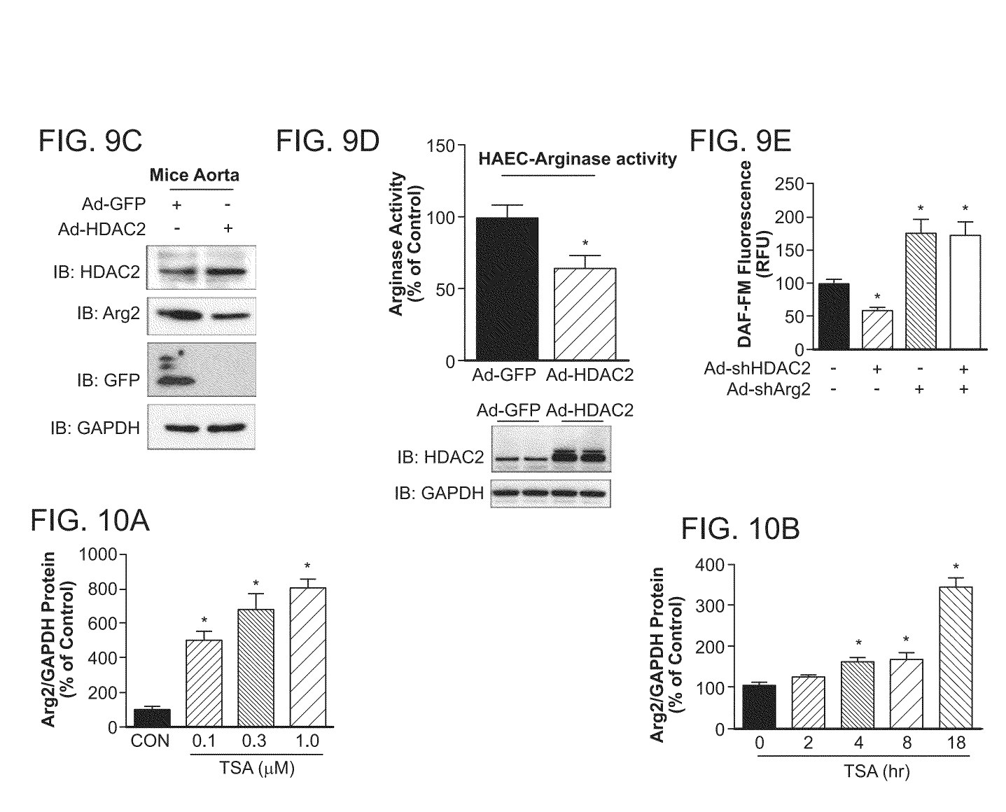

It was next determined whether HDAC2 overexpression could be used as a strategy for reversing vascular dysfunction because of atherogenic stimuli such as OxLDL. Isolated mice aortic rings were transduced with either HDAC2 or GFP (control) adenoviruses, and incubated with or without OxLDL (50 μg/mL) for 48 hours. As shown in FIG. 9A, OxLDL significantly impaired endothelial-dependent vascular relaxation (acetylcholine study) compared with the untreated controls (Eₘₐₓ 52.1±3.1% versus 93.0±2.8%; P<0.0001; n=6), suggesting that endothelial function is compromised in this group. The dose-response effect of SNP on OxLDL-incubated aortic rings was not significantly different from that in the untreated controls, suggesting that OxLDL does not affect the endothelial-independent component of blood vessel reactivity. Furthermore, mice aortic rings overexpressing HDAC2 (FIG. 9C and FIG. 14E) exhibited significantly improved acetylcholine-dependent vascular relaxation despite OxLDL exposure compared with GFP-overexpressing aortic rings exposed to OxLDL (Eₘₐₓ78.4±5.2% versus 52.1±3.1%; P<0.0001; n=4-6) (FIG. 9A). There was no significant difference in SNPinduced vascular relaxation between these groups (FIG. 9B). Consistent with studies in cultured HAECs, Arg2 expression was significantly attenuated in mice aortas overexpressing HDAC2 (FIG. 9C, second panel). Increased expression of HDAC2 in HAECs also decreased the total arginase activity, and it was determined that this activity was predominantly due to Arg2.

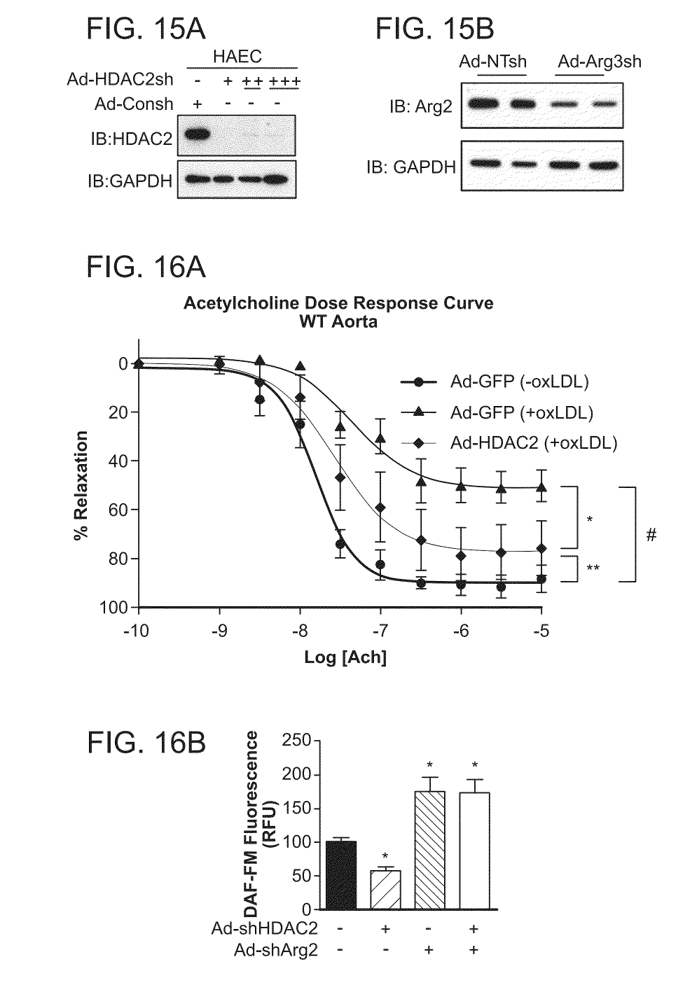

It was next determined whether increased expression of Arg2 as observed with knockdown of HDAC2 affects NO production in HAECs. Indeed, HDAC2 shRNA significantly attenuated NO production as determined with DAF-FM DA fluorescence, and this was rescued above the control levels in the presence of Arg2 shRNA (FIG. 9E). NO production was also significantly augmented in HAECs where only Arg2 was knocked down. The effectiveness of Arg2 and HDAC2 shRNAs in HAECs is demonstrated in FIG. 15A and FIG. 15B.

### Example 10

### Histone Deacetylase 2 Reverses Endothelial Dysfunction and Atherosclerosis

Increased Arginase 2 (Arg2) expression reciprocally dysregulates endothelial nitric oxide synthase (eNOS), resulting in impaired endothelial function—a critical event in the development of atherosclerosis. A regulatory mechanism for arginase 2 transcription and its role in vascular endothelial homeostasis whereby histone deacetylase 2 (HDAC2) concomitantly decreases Arg2 expression and enhances Nitric Oxide production in endothelial cells (EC) to provide protection against the endothelial injury stimulus oxidized low-density lipoprotein (OxLDL) is described herein. The goal of this study was to establish the physiological functions of HDAC modulation of ARG2, and the consequences of pathophysiologic dysregulation of this pathway.

Isolated mice aortic rings were transduced with either GFP or HDAC2 adenoviruses (100 MOI) for 24 hrs and incubated for additional 24 hrs in the presence or absence of OxLDL (50 μg/mL). After 48 hours, dose-response effects of Ach on vascular relaxation were determined using wire myography (FIG. 16A). NO production was determined using the DAF-FM DA fluorescence assay in HAEC that were transduced with either Ad-Arg2shRNA or Ad-HDAC2shRNA alone, or with both reagents in combination (FIG. 16B). This demonstrates that augmented HDAC2 expression provides protection against OxLDL-mediated endothelial dysfunction.

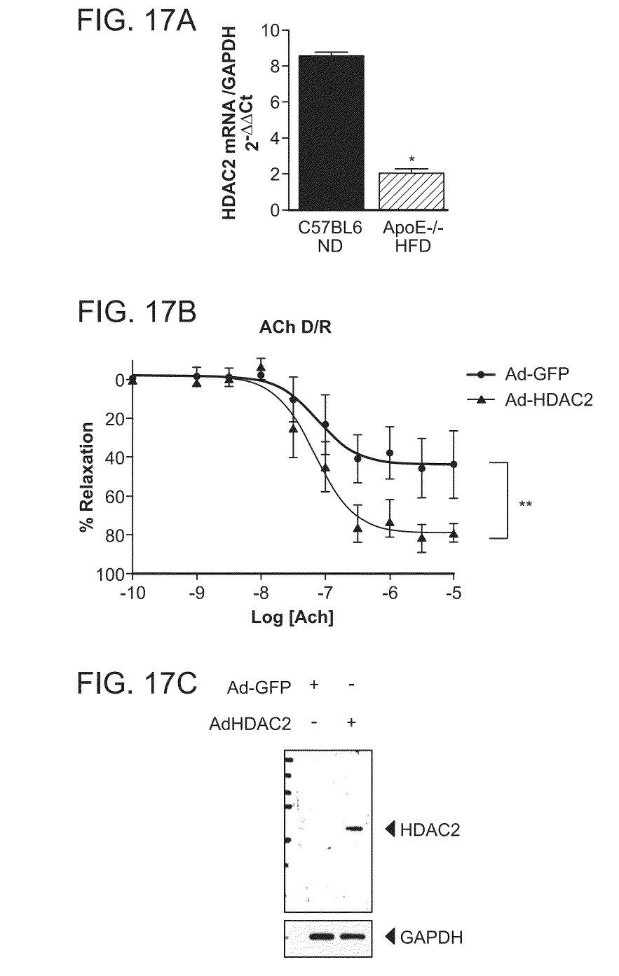

Total RNA was extracted from intact aortas isolated from C57BL6 (WT) or ApoE−/− mice fed an atherogenic diet. cDNA was synthesized followed by semiquantitative RT PCR using taq polymerase with HDAC2- and GAPDH-specific primers (FIG. 17A). Dose-response to Ach in aortic rings isolated from ApoE−/− mice with an atherogenic diet that were subjected to 3 injections of either adenoviral HDAC2 or GFP (control) is shown in FIG. 17B. HDAC2 expression in isolated aortas from FIG. 17B is depicted in FIG. 17C. This demonstrates that in vivo HDAC2 gene transfer improves vascular endothelial function in ApoE KO mice fed a high fat diet.

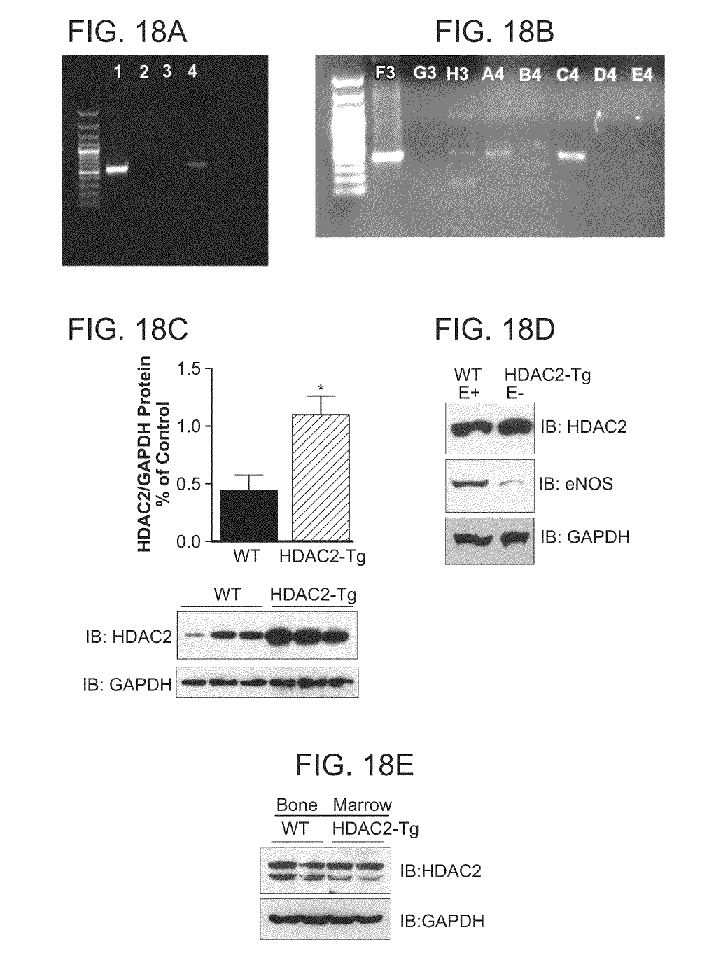

The generation and characterization of hHDAC2 transgenic mice was performed. FIG. 18A and FIG. 18B shows primers specific to human HDAC2 were used for PCR analysis using Genomic DNA isolated from the tails of the following mice as templates: for FIG. 18A C57BL6 (lane 2), non-transgenic littermates (lane 3), and an HDAC2 transgenic founder (lane 4); a transgenic DNA construct that was injected into the murine ooytes was used as positive control template (lane 1); for FIG. 18B F3 positive control; G3 through E4, HDAC2 positive pups exhibiting high (C4), medium (A4), and low (E4) expression levels. FIG. 18C and FIG. 18D shows snap frozen isolated aortas from WT and HDAC2-Tg mice (FIG. 18C), and Aortas from WT and endothelium-denuded (E-) aortas from HDAC2-Tg mice (FIG. 18D) were subjected to western blotting using anti-HDAC2, eNOS and GAPDH (loading control) antibodies. FIG. 18E shows bone marrow was harvested from femurs, and subjected to western blotting using anti-HDAC2 and GAPDH antibodies. HDAC2-Tg mice were generated using the construct scheme depicted in FIG. 28.

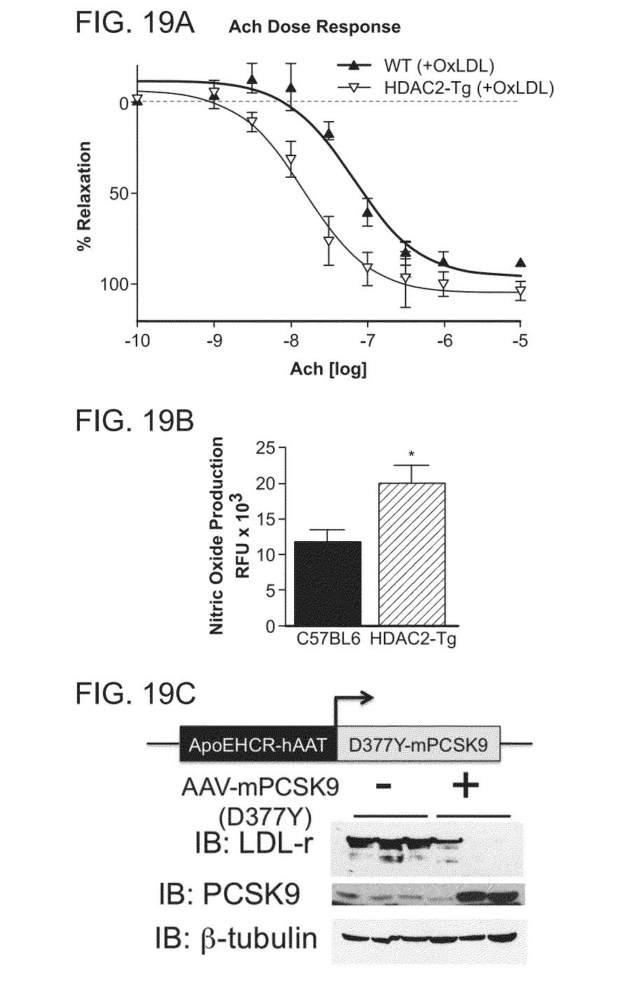

Isolated aortic rings from WT and HDAC2-Tg mice were incubated with OxLDL (50 μg/mL) for 48 hours and dose-response to Ach was determined (FIG. 19A). Isolated aortas from WT or HDAC2-tg mice were loaded with DAF2 FM for NO measurement (FIG. 19B). C57BL6 mice were injected with adeno-associated viruses encoding murine PCSK9 D377Y cDNA with a liver-specific promoter. Liver lysates were immunoblotted for PCSK9& LDL-r (FIG. 19C). This demonstrates that transgenic mice with endothelial-specific HDAC2 overexpression (HDAC2-Tg) exhibit enhanced endothelial NO production and endothelial-dependent vasorelaxation. HDAC2-Tg mice were generated using the construct scheme depicted in FIG. 28.

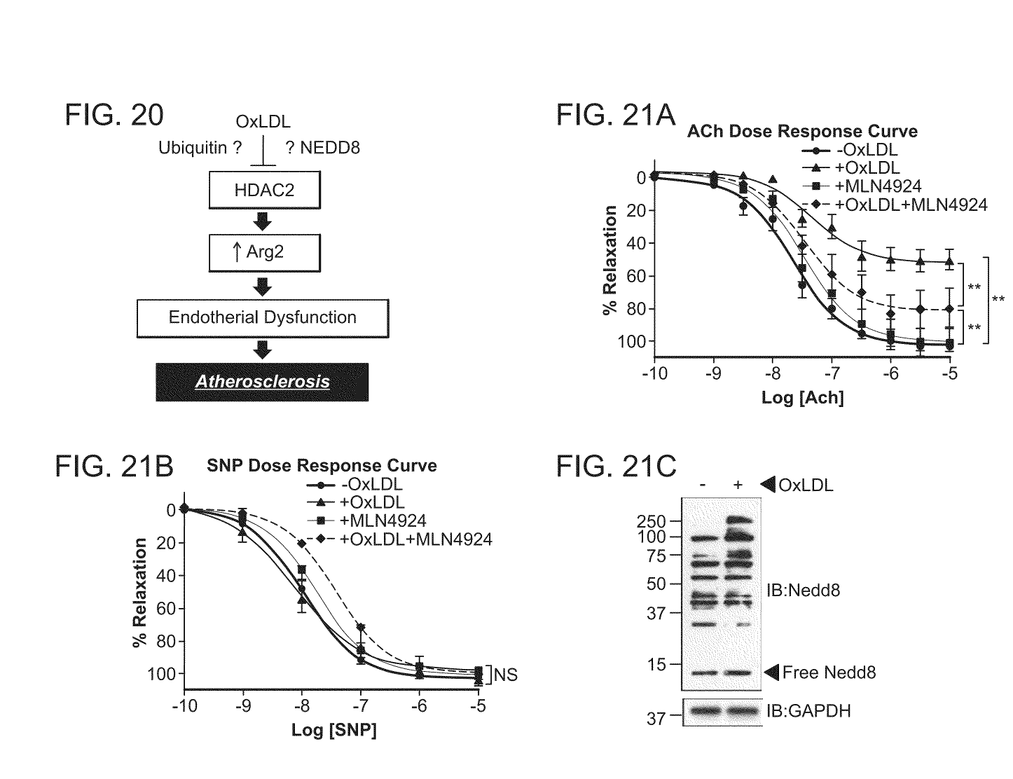

A proposed pathway for HDAC2 modulation of endothelial Arg2 and resulting endothelial dysfunction is depicted in FIG. 20. OxLDL downregulates HDAC2 expression via NEDD8- and/or Ubiquitin-mediated proteasomal degradation. A drop in HDAC2 levels leads to transcriptional upregulation of Arg2, L-arginine depletion, eNOS uncoupling, and EC dysfunction.

ApoE KO mice fed a high fat diet for 12 weeks exhibited significant lower levels of HDAC2 in their aortas. HDAC2 gene transfer achieved via tail vein injection of adenoviruses encoding HDAC2 followed by continuing HF diet reversed endothelial-dependent vascular relaxation. Mice expressing human HDAC2 in their endothelium as controlled by constitutive expression via the Tie2 promoter were successfully generated and are being phenotyped. HDAC2-Tg mice are backcrossed with ApoE KO mice or injected with PCSK9 AAV followed by a HF diet regimen for 4 months. Vascular function and atheogenesis progression are determined in these mice and compared to those of APOE KO and C5BL6 mice that are injected with PCSK9AAV.

### Example 11

### NEDDylation Promotes Endothelial Dysfunction: a Role for HDAC2

Described herein is a role for HDAC2 in the transcriptional regulation of endothelial genes and vascular function. As described herein, HDAC2 reciprocally regulates the transcription of Arginase 2, which is itself a critical modulator of endothelial function via eNOS. Moreover, HDAC2 levels are decreased in response to the atherogenic stimulus OxLDL via a mechanism that is apparently dependent upon proteasomal degradation. NEDDylation is a post-translational protein modification that is tightly linked to ubiquitination and thereby protein degradation. Changes in NEDDylation may modulate vascular endothelial function in part through alterations in the proteasomal degradation of HDAC2. In HAEC, OxLDL exposure augmented global protein NEDDylation. Pre-incubation of mouse aortic rings with the NEDDylation activating enzyme inhibitor, MLN4924, prevented OxLDL-induced endothelial dysfunction. In HAEC, MLN enhanced HDAC2 abundance, decreased expression and activity of Arginase 2, and blocked OxLDL-mediated reduction of HDAC2. Additionally, HDAC2 is a substrate for NEDD8 conjugation and this interaction was potentiated by OxLDL. Further, HDAC2 levels were reciprocally regulated by ectopic expression of NEDD8 and the de-NEDDylating enzyme SENP8. The results presented herein indicate that the observed improvement in endothelial dysfunction with inhibition of NEDDylation activating enzyme is likely due to an HDAC2-dependent decrease in Arginase 2. NEDDylation activating enzyme is a target in endothelial dysfunction and atherogenesis.

The considerable burden of atherosclerotic and other vascular diseases on health, cost, and productivity in both western and developing countries continues to fuel investigation into pathogenic mechanisms that are amenable to modulation or ablation by new therapies (Kones R. 2013 Vasc Health Risk Manag, 9:617-70). The presence of proteasomal degradation across the spectrum of cellular, tissue, and organ system contexts has ignited a focus on modulation of proteasome dynamics for a wide variety of clinical applications ranging from cancer to autoimmune diseases (Xolalpa W et al. 2013 Curr Pharm Des, 19:4053-93). Recent studies highlight the importance of NEDDylation for a subset of ubiquitin-dependent protein degradation (Oved S, et al. 2006 J Biol Chem, 281:21640-51), thereby offering a therapeutic target that narrows the spectrum of target proteins and limits impact on off-target protein turnover events.

A specific role for HDAC2 in the transcriptional suppression of Arginase 2 was identified as described herein—Arginase 2 is an uncoupling inhibitor of eNOS. OxLDL decreases the level of HDAC2 protein in vascular endothelium, and demonstrated that the expression of HDAC2 defends vascular function in the setting of oxidative injury (Pandey D, et al. 2014 Arterioscler Thromb Vasc Biol, 34:1556-66). As described herein, the NEDDylation activating enzyme (NAE) inhibitor, MLN4924, blocks OxLDL-mediated vascular dysfunction via upregulation of HDAC2 abundance. This data indicates that NEDDylation is a key regulatory input for HDAC2 and vascular homeostasis.

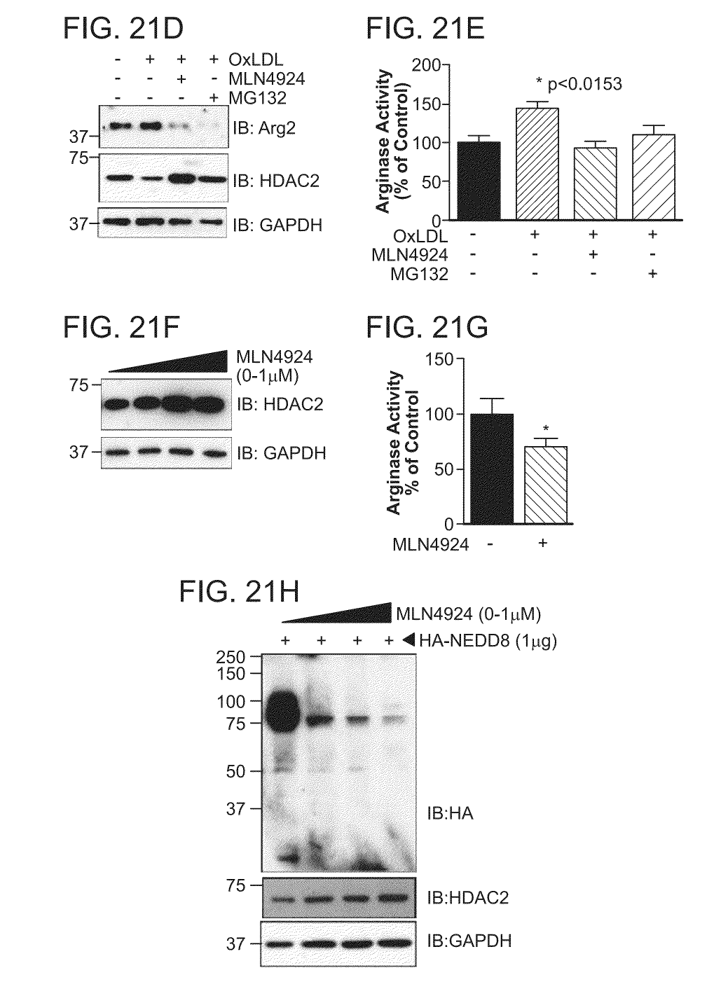

As described in detail below, inhibition of NEDDylation provides protection against OxLDL-mediated vascular endothelial dysfunction. To better understand the link between NEDDylation and vascular function, ex-vivo vascular reactivity experiments were performed in isolated mice aortas incubated with MLN4924 (1 μM) prior to OxLDL (50 μg/mL) exposure. Impairment of endothelial-dependent vascular relaxation by OxLDL was assessed using acetylcholine dose response curves (FIG. 21A). Substantial rescue of endothelial function was demonstrated in aortas pretreated with MLN4924 prior to OxLDL stimulation, whereas MLN4924 alone had negligible effect (FIG. 21A). In contrast, endothelial-independent vascular relaxation as assessed using sodium nitroprusside was unaffected by either OxLDL or MLN4924 (FIG. 21B).

Exposure of HAEC to OxLDL (50 μg/mL) for 24 h increased global protein NEDDylation (FIG. 21C). Furthermore, pretreatment of OxLDL-exposed HAEC with either the NEDDylation inhibitor-MLN4924 or the proteasome inhibitor-MG132 rescued HDAC2 levels (FIG. 21D). The results presented herein indicate that oxidative injury to the vascular endothelium triggers HDAC2 degradation via NEDDylation and ubiquitination.

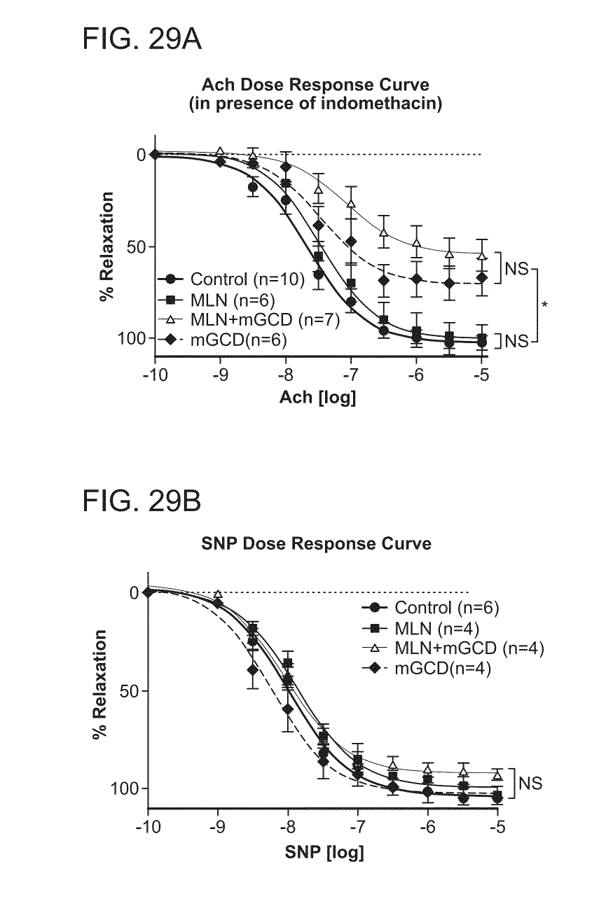

Arg2 has been identified as a major culprit in vascular endothelial dysfunction induced by OxLDL and the dominant arginase isoformin aortic endothelium (Pandey D, et al. 2014 Circ Res, 115:450-9). As shown in FIGS. 21D and 21E, OxLDL-induced increases in the abundance and activity of endothelial arginase were abolished withMLN4924 and MG132. MLN4924 was then used to determine whether HDAC2 and arginase levels were modulated by NEDDylation in HAEC in the absence of OxLDL. A robust dose-dependent increase in HDAC2 protein expression was observed (FIG. 21F), as was decreased arginase activity (FIG. 21G). MLN4924 did not rescue vascular endothelial dysfunction that resulted from the inhibition of class I HDACs (FIGS. 29A and 29B). The NAE inhibitorMLN4924 attenuated the quantity of NEDDylation conjugates in a dose-dependent fashion in HAEC that expressed ectopic HA-tagged-NEDD8 (FIG. 21H).

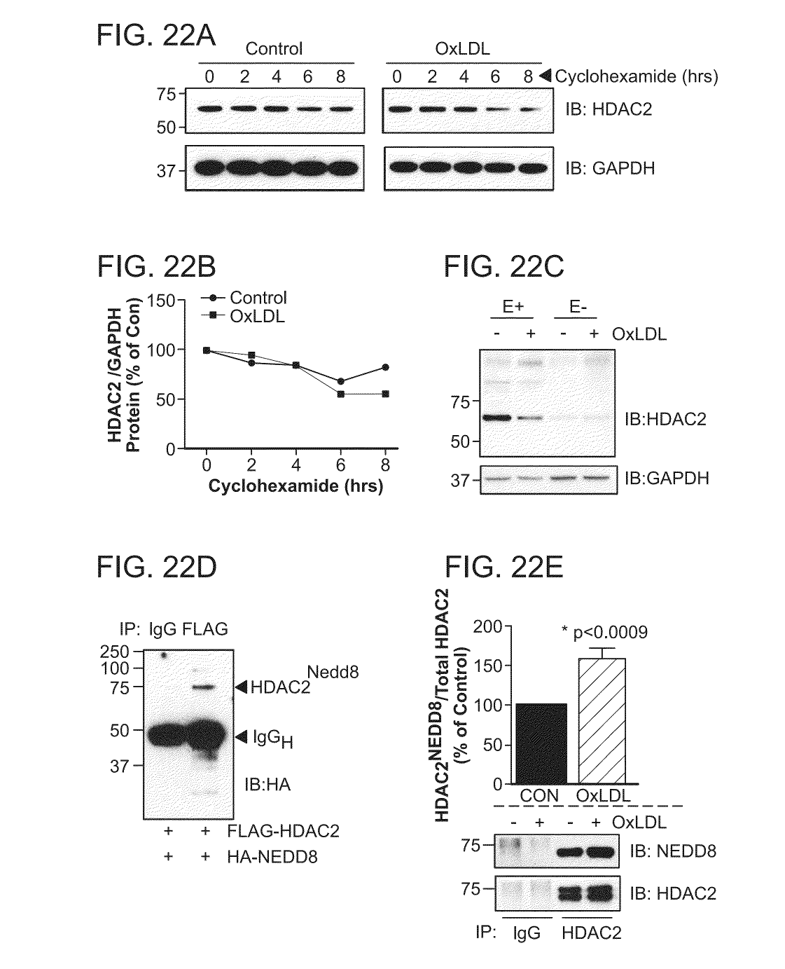

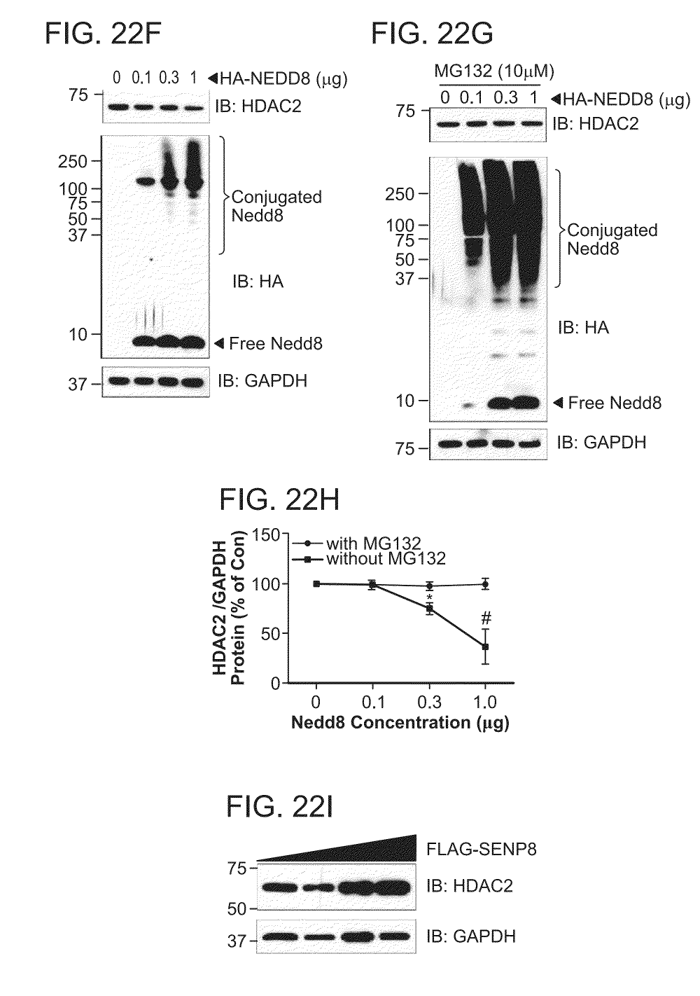

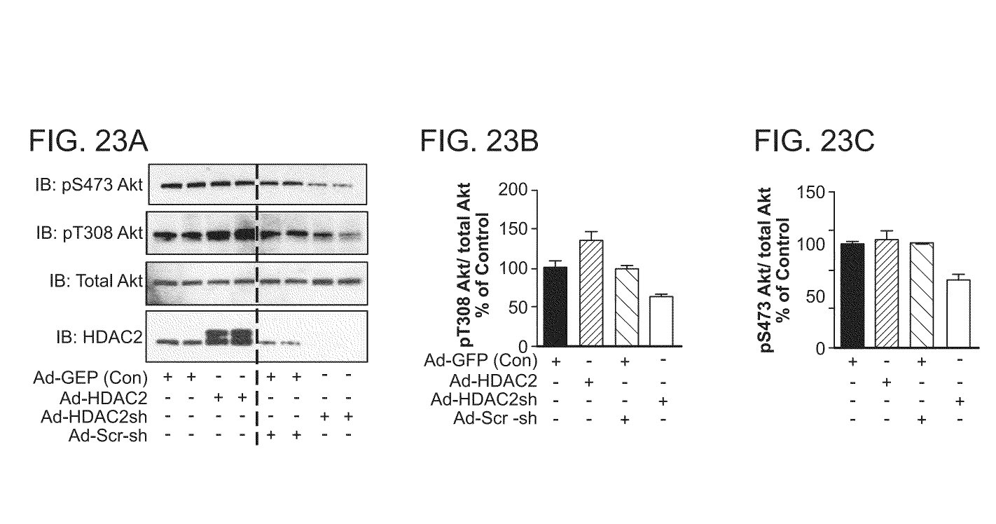

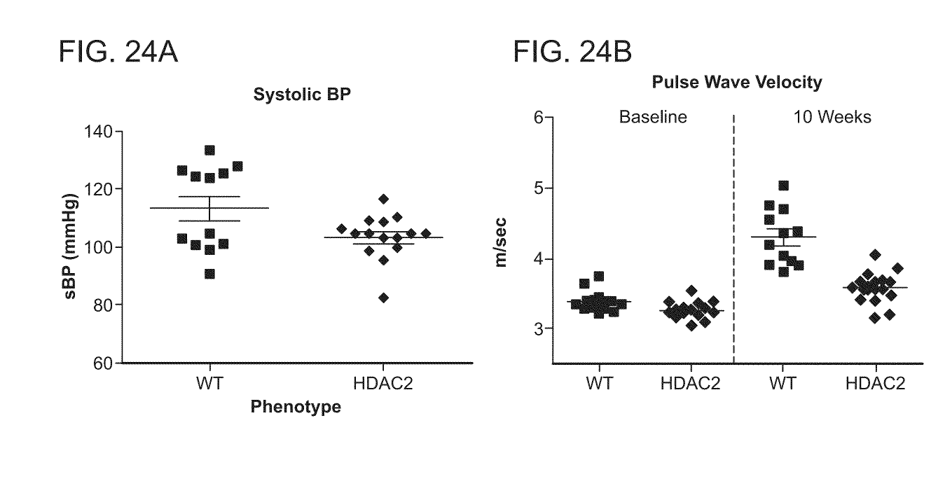

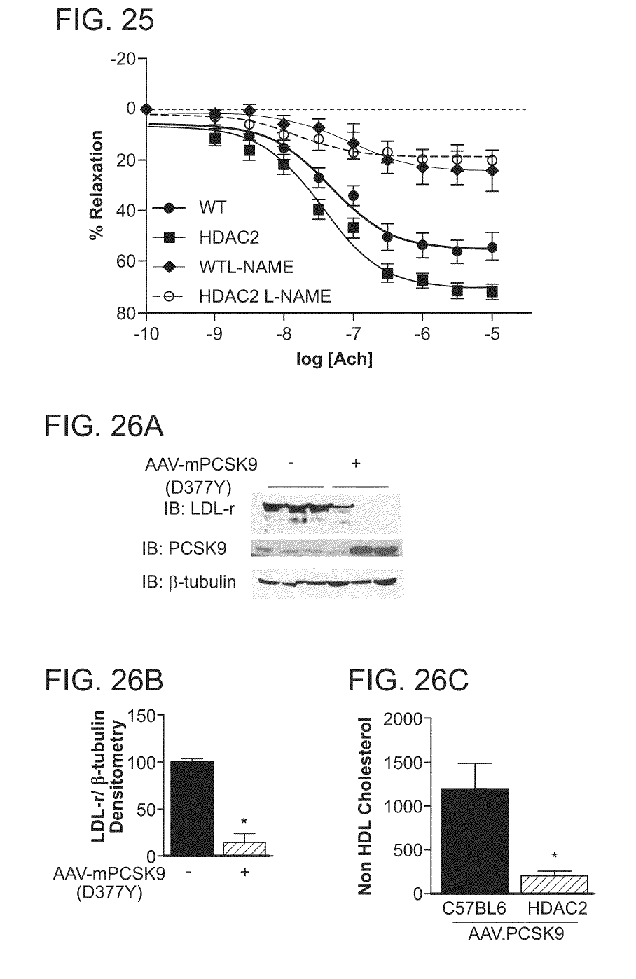

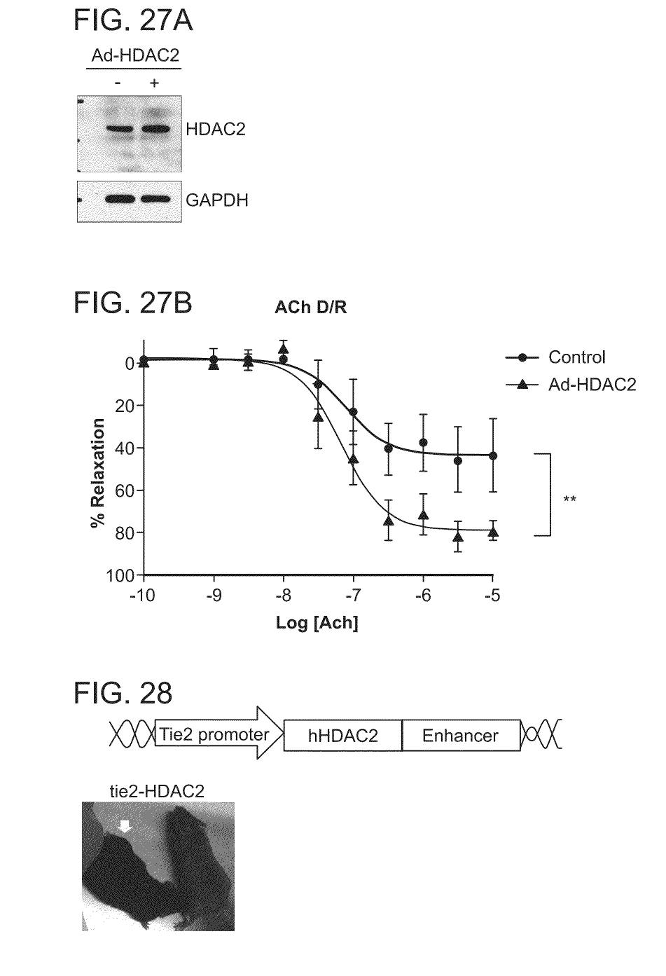

NEDDylation of HDAC2 hastens its clearance from vascular endothelium. The effect of OxLDL on HDAC2 protein stability was assessed with a cycloheximide chase experiment that demonstrated a larger drop in HDAC2 levels at 6 and 8 h in the presence of OxLDL (FIGS. 22A and 22B).

Endothelium-denuded murine aortas had dramatically reduced levels of HDAC2 that were unchanged by OxLDL exposure (FIG. 22C). These results demonstrate that endothelium accounts for the majority of HDAC2 protein expression in large blood vessels, and that it is the nidus for OxLDL-mediated changes in HDAC2 levels.

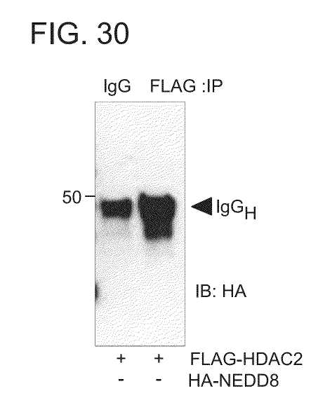

The possibility that HDAC2 is a NEDD8 substrate was then tested. HAEC were co-transfected with FLAG-tagged-HDAC2 and HA-tagged-NEDD8. Immunoprecipitation with anti-FLAG antibody yielded an ˜70 kDA band corresponding to the molecular weight of NEDDylated HDAC2 upon immunoblotting with antibody against HA (FIG. 22D). In cells that were not transfected with HA-NEDD8, migrating higher molecular weight bands were not observed (FIG. 30). The effect of OxLDL on HDAC2 NEDDylation was assessed by immunoprecipitation of lysates from OxLDL-exposed HAEC with anti-HDAC2 antibody followed by immunoblotting for NEDD8 or HDAC2. OxLDL treatment augmented the abundance of NEDDylated HDAC2 (FIG. 22E, lower panel) and the densitometric analysis showed that increase was almost 60% as compared to control (FIG. 22E, upper panel).

To determine the role of NEDDylation in ubiquitin-mediated proteasomal degradation of HDAC2, HAEC were transfected with HA-NEDD8 cDNA. Expression of HA-NEDD8 decreased HDAC2 levels in a dose-dependent manner (significant effect was observed only at 0.3 and 1 μg of NEDD8 transfected cells), and this effect was abolished by MG132 (FIGS. 22F-H). These data demonstrate that there is a reciprocal relationship between NEDDylation and HDAC2 abundance, and this was corroborated by data from transfection of HAEC with the deNEDDylase SENP8 in which blockade of NEDDylation-mediated clearance increased HDAC2 in a dose-dependent manner (FIG. 22I).

Protein NEDDylation, the covalent conjugation of NEDD8 substrate to target proteins, is emerging as a critical post-translational protein modification process. Evidence suggests that NEDDylation is tightly associated with ubiquitination and the regulation of cellular protein turnover (Oved S, et al. 2006 J Biol Chem, 281:21640-51), although NEDDylation has other functions as well. A paucity of literature exists with regard to NEDDylation and the regulation of vascular function, but two recent studies have shown the inhibition of NEDDylation to be effective in attenuating NFkB-mediated proinflammatory cytokine release by LPS-activated macrophages and by human microvascular endothelial cells (Ehrentraut S F, et al. 2013 J Immunol, 190:392-400) (Chang F M, et al. 2012 J Biol Chem, 287:35756-67). Furthermore, knockdown of the SENP8 NEDDylase in human microvascular endothelial cells blocks LPS-mediated activation of NFKB and HIFla (Ehrentraut S F, et al. 2013 J Immunol, 190:392-400).

Described herein is the determination of whether NEDDylation might regulate endothelial function, and whether this regulation might be dependent on HDAC2, a transcriptional regulator of Arg2. Arg2 is a critical reciprocal regulator of NOS and NO production and thereby endothelial dysfunction (Pandey D, et al. 2014 Circ Res, 115:450-9). Additionally, Arg2 is transcriptionally regulated in an HDAC2-dependent manner (Pandey D, et al. 2014 Arterioscler Thromb Vasc Biol, 34:1556-66). Described herein is evidence which demonstrates that the atherogenic stimulus OxLDL leads to a significant increase in global protein NEDDylation in aortic intima, and that inhibition of NEDDylation improves OxLDL-induced endothelial dysfunction. This improvement is associated with an increase in the abundance of HDAC2 and a decrease in Arg2 activity and abundance. A connection between NEDDylation, regulation of HDAC2 abundance by proteasomal degradation, and Arginase 2 a critical regulator of endothelial NO and endothelial function has been established. These data are supported by a report that HDAC2 levels were reduced in human atherosclerotic vessels (Dje N′Guessan P, et al. 2009 Arterioscler Thromb Vasc Biol, 29:380-6).

Protein NEDDylation enhances ubiquitination by a number of mechanisms. First, NEDD8 attachment can induce conformational changes of its targets. For example NEDDylation of cullins stimulates their ubiquitin E3 activity enhancing ubiquitination (Kawakami T, et al. 2001 EMBO J, 20:4003-12). Second, NEDDylation can block interaction with the cullin inhibitor CAN D1 which preferentially binds to un-NEDDylated cullins (Goldenberg S J, et al. 2004 Cell, 119:517-28). Finally, NEDDylation can stimulate the recruitment of NEDD8-interacting proteins. For example, NEDD8 can interact directly with the ubiquitin E2 Ubc4 and this interaction has been proposed to participate in the activation of cullin-based ubiquitin ligases (Sakata E, et al. 2007 Nat Struct Mol Biol, 14:167-8).

It is also possible that HDAC2 is itself a target for NEDDylation, and that this process directly enhances the proteasomal degradation of HDAC2. Indeed, HDAC2 has been implicated as a NEDD8 substrate in a recent mass spectrometry screen of NEDD8-associated proteins in HEK-293 cells (Jones J, et al. 2008 J Proteome Res, 7:1274-87). The data described herein demonstrate dose-dependent increases in the abundance of HDAC2 in the presence of the SENP8 deNEDDylase, and decreases in levels of HDAC2 in HAEC with the addition of NEDD8. These data also indicate that HDAC2 is directly NEDDylated by NEDD8 in endothelial cells. Although the mechanism still requires full elucidation, it is clear that inhibition of NEDDylation leads to less HDAC2 degradation, increased HDAC2 abundance, and improved endothelial function despite concomitant exposure to OxLDL. Thus, the NEDD8 activating enzyme may represent a focus for therapeutic drug design in atherosclerosis and other vascular diseases in which the endothelium is a principal target.

Transcriptional regulation of pathways that determine nitric oxide availability in vascular endothelium is a process with both broad and sustained effects. The recently described role of HDAC2 in the transcriptional regulation of endothelial Arginase 2 offers a means of defense against the pathogenesis of vascular endothelial dysfunction and its devastating contribution to morbidity and mortality in a host of vascular diseases. The results presented herein demonstrate a direct link between oxidative injury to vascular endothelium and NEDDylation specific acceleration of HDAC2 turnover—an insight that expands the understanding of links between specific mechanisms of cellular injury and proteasomal degradation and pave the way for targeted treatments for vasculopathies.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating or preventing endothelial dysfunction or an endothelial dysfunction-associated condition in a subject, the method comprising:
identifying a subject having or at risk of developing endothelial dysfunction or an endothelial dysfunction-associated condition;
administering to the subject an effective amount of an agent that reduces arginase 2 (Arg2) transcription or activity, thereby treating or preventing endothelial dysfunction or an endothelial dysfunction-associated condition in the subject.

2. The method of claim 1, wherein the endothelial dysfunction-associated condition comprises atherosclerosis, chronic obstructive pulmonary disease (COPD), vasculitis, hypertension or a local or systemic inflammatory response.

3. The method of claim 2, wherein the hypertension comprises pulmonary hypertension.

4. The method of claim 2, wherein the vasculitis comprises pulmonary vasculitis.

5. The method of claim 1, wherein the agent that reduces Arg2 transcription or activity comprises histone deacetylase (HDAC) or an HDAC agonist.

6. The method of claim 5, wherein the HDAC comprises an HDAC polynucleotide or HDAC polypeptide.

7. The method of claim 6, wherein the HDAC polynucleotide comprises HDAC complementary deoxyribonucleic acid (cDNA) or HDAC messenger ribonucleic acid (mRNA).

8. The method of claim 5, wherein the HDAC comprises HDAC2.

9. The method of claim 8, wherein the HDAC agonist comprises a small molecule agonist of HDAC2.

10. The method of claim 9, wherein the small molecule agonist of HDAC2 comprises a methylated xanthine (methylxanthine) or curcumin.

11. The method of claim 10, wherein said methylxanthine is administered at a dose of 1 mg/kg/day-1 g/kg/day.

12. The method of claim 10, wherein the methylxanthine comprises caffeine, aminophylline, 3-isobutyl-1-methylxanthine (IBMX), paraxanthine, pentoxifylline, theobromine, theophylline (1,3-dimethylxanthine), or curcumin.

13. The method of claim 1, wherein endothelial nitric oxide (NO) production is increased.

14. The method of claim 1, wherein said subject is a human.

15. The method of claim 1, wherein said agent is administered orally, intravenously, intramuscularly, systemically, subcutaneously or by inhalation.

16. The method of claim 7, wherein said HDAC polynucleotide is administered via a vector.

17. The method of claim 16, wherein the vector comprises an adenoviral vector.

18. The method of claim 2, wherein the vasculitis is systemic, pulmonary, or cerebral vasculitis.

19. The method of claim 2, wherein the hypertension is pulmonary or systemic hypertension.

20. The method of claim 2, wherein the local or systemic inflammatory response comprises an inflammatory response during infectious, autoimmune, diabetes-induced vascular dysfunction, or a systemic sepsis-like syndrome.

21. A kit comprising the HDAC2 agonist of claim 9.

22. A method of treating or preventing endothelial dysfunction or an endothelial dysfunction-associated condition in a subject, the method comprising:
identifying a subject having or at risk of developing endothelial dysfunction or an endothelial dysfunction-associated condition;
administering to the subject an effective amount of an agent that reduces NEDDylation activating enzyme (NAE) activity, thereby treating or preventing endothelial dysfunction or an endothelial dysfunction-associated condition in the subject.

23. The method of claim 22, wherein the endothelial dysfunction-associated condition comprises atherogenesis.

24. The method of claim 22, wherein the agent that reduces NEDDylation activating enzyme activity comprises an HDAC agonist, a HDAC, a NAE inhibitor, and/or any combination thereof.

25. The method of claim 24, wherein the NAE inhibitor comprises MLN4924.

26. The method of claim 24, wherein the HDAC comprises an HDAC polynucleotide or HDAC polypeptide.

27. The method of claim 26, wherein the HDAC polynucleotide comprises HDAC complementary deoxyribonucleic acid (cDNA) or HDAC messenger ribonucleic acid (mRNA).

28. The method of claim 24, wherein the HDAC comprises HDAC2.

29. The method of claim 28, wherein the HDAC agonist comprises a small molecule agonist of HDAC2.
